(19) United States
(12) Patent Application Publication
(10) Pub. No.: US 2005/0281857 A1
(43) Pub. Date: Dec. 22, 2005

(54) Methods and reagents for preparing biomolecule-containing coatings

(76) Inventors: Toni M. Heyer, St. Louis Park, MN (US); Dale G. Swan, St. Louis Park, MN (US); Stephen J. Chudzik, St. Paul, MN (US); Joseph A. Chinn, Shakopee, MN (US); Ralph A. Chappa, Prior Lake, MN (US)

Correspondence Address: KAGAN BINDER, PLLC, SUITE 200, MAPLE ISLAND BUILDING, 221 MAIN STREET NORTH, STILLWATER, MN, 55082, US

(21) Appl. No.: 11/138,733

(22) Filed: May 25, 2005

Related U.S. Application Data
(60) Provisional application No. 60/574,316, filed on May 25, 2004.

(51) Int. Cl. A61L 2/00; B05D 3/00

(52) U.S. Cl. 424/423; 427/2.1; 623/11.11

(57) ABSTRACT

Polymeric coatings that include coupled biomolecules are formed on the surface of articles, such as medical devices. A coated layer that includes a synthetic polymer including a reactive group is formed on a surface and then a biomolecule is attached to the reactive group. The synthetic polymer can be an acrylate polymer or an amine-containing polymer having pendent photoreactive groups.

### CROSS-REFERENCE TO RELATED APPLICATIONS

The present non-provisional Application claims the benefit of commonly owned provisional Application having Ser. No. 60/574,316, filed on May 25, 2004, and entitled METHODS AND REAGENTS FOR PREPARING BIOCOMPATIBLE COATINGS, which application is incorporated herein by reference in its entirety.

### FIELD OF THE INVENTION

The invention generally relates to the immobilization of biological material using synthetic polymers. The invention also relates to preparing biocompatible surfaces on medical devices.

### BACKGROUND OF THE INVENTION

Many different approaches have been used for the attachment of biomolecules to target surfaces. Attachment technologies have been useful in many areas, including biosensors, such as glucose sensors, immunodiagnostic reagents and strips, protein and nucleic acid microarrays, purification apparatus, cell analysis technologies, including flow cytometry, etc. In many cases these approaches have been aimed at preserving the activity or function of the biomolecule during and after the attachment process has been performed. This, however, can be a difficult process, as chemical groups present in active sites on proteins and polysaccharides often participate in reactions that result in covalent bonding between the biomolecule and the substrate. If active site residues participate in chemical attachment of the biomolecule to the surface its activity or function can be compromised or lost.

In addition to challenges that are presented in preserving biomolecule activity or function, another consideration in the art of the attachment of biomolecules to target surfaces involves the properties of the target surface. In many cases, an unmodified target surface may not be suitable for biomolecule attachment. For example, the biomolecule may not spread out properly on the surface, or the surface may not provide a suitable reactive target for the contemplated attachment chemistries.

More recently, biomolecule attachment to surfaces has been facilitated by intermediate polymeric coatings. However, there is a continued need to provide new polymeric reagents and coating process for the improved biomolecule attachment to target surfaces. This can be seen in the need to provide biocompatible surfaces to medical devices, that is, where a biocompatible molecule such as heparin is indirectly attached to the surface of a medical device.

Improved compatibility with blood is a desired feature for a variety of medical devices that contact blood during clinical use. The materials used for manufacture of medical devices are not inherently compatible with blood and its components, and the response of blood to a foreign material can be aggressive, resulting in surface induced thrombus (clot) formation. This foreign body response can in turn impair or disable the function of the device and, most importantly, threaten patient health. It is often desirable to modify the surface of medical devices to provide a biocompatible surface, to minimize or avoid such adverse foreign body responses.

As used herein, a surface of a medical device is characterized as “biocompatible” if it is capable of functioning or existing in contact with biological fluid and/or tissue of a living organism with a net beneficial effect on the living organism. Long-term biocompatibility is desired for the purpose of reducing disturbance of a host organism. One approach to improved biocompatibility for medical device surfaces is to attach various biomolecules such as antithrombogenic agents, anti-restenotic agents, cell attachment proteins, growth factors, and the like, to the surface of the device. For example, antithrombogenic agents can reduce the generation of substances as part of the clotting cascade, antirestenotic agents can reduce generation of aggressive scar tissue growth around the device, while cell attachment proteins can contribute to the growth of a layer of endothelial cells around the device.

Several benefits can be provided by biocompatible medical device surfaces. For example, such surfaces can increase patient safety, improve device performance, reduce adherence of blood components, inhibit blood clotting, keep device surfaces free of cellular debris, and/or extend the useable lifetime of the device.

One biomolecule that has been utilized to improve biocompatibility of medical device surfaces is heparin. Heparin is a pharmaceutical that has been used clinically for decades as an intravenous anticoagulant to treat inherent clotting disorders and to prevent blood clot formation during surgery and interventional procedures. Heparin molecules are polysaccharides with a unique chemical structure that gives them specific biological activity. When heparin is immobilized onto the surface of a medical device material, it can improve the performance of the material when in contact with blood in several ways: 1) it can provide local catalytic activity to inhibit several enzymes critical to the formation of fibrin (which holds thrombi together); 2) it can reduce the adsorption of blood proteins, many of which lead to undesirable reactions on the device surface; and 3) it can reduce the adhesion and activation of platelets, which are a primary component of thrombus.

In addition to heparin, other biomolecules that can be provided on a medical device to improve biocompatibility include extracellular matrix (ECM) proteins or ECM peptides derived from these proteins. Surfaces modified with appropriate proteins or peptides are less likely to be recognized as foreign than the original device surface and will promote the attachment and overgrowth of specific desirable cell types.

In addition to the technical challenges that are present in the preparation of a biocompatible surface, another challenge involves improving the coating technology to provide cost-effective reagents and methods that can be used in the preparation of a wide variety of medical devices that have biomolecular coatings. Some coating processes are labor intensive and/or require the use of expensive reagents (for example, many biomolecule or biocompatible agents are expensive to produce). While these processes might be economically justified in the preparation of medical devices or items that are sold at a high cost, to carry out these processes in the production of medical devices that are sold at a medium or lower cost is economically unrealistic. Nonetheless, there is a demand for medium or lower cost medical devices or items that have biocompatible coatings.

### SUMMARY OF THE INVENTION

The invention generally relates to reagents and methods for providing a coating to the surface of an article, the coating including a polymeric material and a biomolecule that is coupled to the polymeric material. The coating is arranged to stably present the biomolecule on the surface of the coating, so the biomolecule can interact with components that are placed in contact with the coated surface. In the present invention, polymeric material is utilized to facilitate the immobilization of a biomolecule on the surface of an article. The polymeric material that is used to form a coated layer on a surface of the article is either (a) a polymer that can be adhered to the surface of the article, or (b) a polymer that can be covalently bound to the surface via a latent reactive group. In some aspects of the invention, a biomolecule is coupled directly to the polymeric material that is used to form the coated layer. First and second reactive groups are present on the polymeric material and the biomolecule, respectively, to provide a coupling mechanism.

In other aspects of the invention, the coating includes a first polymeric material and a second polymeric material having a first reactive group, wherein the second polymeric material can be mixed with and immobilized in the first polymeric material to form a coated layer. A biomolecule having a second reactive group is reacted with the first reactive group of the second polymeric material thereby coupling the biomolecule to the coated layer.

The reagents and methods of the invention offer many advantages for the preparation of these types of coatings and therefore can be used in a wide range of technologies such as medical devices, biosensors, immunoassay systems; cell biology articles; cell culture articles; chromatography and separation systems; filters and filtration equipment; and microarray articles.

The methods of the invention advantageously allow a biomolecule-containing coating to be formed on a surface of an article in a minimal number of steps. This greatly reduces the throughput time for the fabrication of coated articles such as medical devices and can result in a substantial cost savings as many reagents and steps that might typically be attempted in fabrication of these coated medical devices are not necessarily required.

The compositions and methods of the invention can provide coatings that are readily prepared and that provide biomolecule-associated properties. Any suitable biomolecule can be coupled to the polymeric material via the reactive pair. The biomolecule can be a larger molecule, such as a polymer that includes amino acid, nucleic acid, or saccharide monomeric units; or a smaller molecule that is non-polymeric, for example, a small synthetically prepared or naturally derived molecule. In preferred aspects the biomolecule is a polymer selected from polysaccharides and polypeptides. In yet other aspects, the biomolecule is soluble in a non-aqueous solvent.

In some aspects, the biomolecule is a biocompatible agent and the coating therefore provides biocompatibility. The polymeric material can be arranged to allow the biomolecule to be presented on the surface of the coated article at a high density, thereby improving the properties of the device.

In some aspects, the biomolecule can provide a biocompatible surface to the coated article, such as a medical device. For example, a medical device with a biocompatible coating can reduce effects that may associated with placing a foreign object in contact with blood components, such as the formation of thrombus or emboli. Useful biocompatible agents can have antirestenotic effects, such as antiproliferative, anti-platelet, and/or antithrombotic effects.

Therefore, the invention also relates to coatings that include a biocompatible agent coupled to a polymeric material using the methods described herein. According to the invention, particularly useful biocompatible agents are polysaccharides that can be selected from mucopolysaccharides such as heparin, hyaluronic acid, chondroitin, keratan, and dermatan. In preferred embodiments the biocompatible agent is heparin, which includes heparin derivatives, sodium heparin, low molecular weight heparin, high affinity heparin, and the like. In yet other aspects, heparin that is soluble in a non-aqueous solvent is used, such as benzalkonium heparin.

In other aspects, the invention provides a coating having a heparin activity of 5 mU/cm² or greater, 10 mU/cm² or greater, or 15 mU/cm² or greater.

The biomolecule can be stably presented on the surface of the substrate by coupling the polymeric material to the biomolecule through use of a reactive pair. The reactive pair consists of a first reactive group pendent from the polymeric material and a second reactive group pendent from the biomolecule, wherein the first group and second group are reactive with each other. For example, a suitable reactive pair would be an electrophilic group/nucleophilic group.

In some aspects the polymeric material comprises a first reactive group that is an amine-reactive group. Useful amine-reactive groups include, but are not limited to, N-oxysuccinimide, isothiocyanate, bromoacetyl, epoxide, and trimethylsilane. The first reactive group is present on the polymeric material in an amount sufficient to promote the coupling of the biomolecule to the polymeric material. In some aspects, greater than about 5%, and preferably in the range of about 5% to about 20%, of the monomeric units of the polymeric material include a first reactive group, such as an amine-reactive group.

In other aspects the polymeric material comprises a first reactive group that is an amine group, and the second reactive group is an amine-reactive group. Polymeric material can be used that provides a polymeric layer with a high density of amine groups.

In some aspects, the biomolecule-containing coating can be prepared using an adherent polymer. This type of polymer can be deposited and stick to a surface without providing substantial additional treatment to make the polymer adhere. The adherent polymer can also provide a suitable stable layer on which a biomolecule can be disposed and coupled via the reaction of a first reactive group included on the adherent polymer and a second reactive group included on the biomolecule. Therefore, in this aspect of the invention, a method for forming a coating includes the steps of (a) disposing a composition comprising an adherent polymer on a surface, the adherent polymer comprising (i) a first reactive group; and (b) disposing a composition comprising a biomolecule comprising a second reactive group that is reactive with the first reactive group, wherein the biomolecule becomes coupled to the adherent polymer. In one aspect step (a) is performed before step (b), while in another aspect, step (a) and step (b) are performed simultaneously.

The adherent polymer can be synthetic or natural. In some aspects the adherent polymer is a synthetic acrylate polymer, examples of which can include alkyl(meth)acrylate and/or aromatic(meth)acrylate polymers. Preferred acrylate polymers include alkyl(meth)acrylates polymers having alkyl chain length from 2 to 8 carbons, for example butyl(meth)acrylate polymers. Therefore, preferred adherent polymers include (i) a first reactive group and (ii) monomeric units selected from the group of alkyl(meth)acrylates and aromatic(meth)acrylates. Suitable adherent polymers can be formed by copolymerizing an acrylate monomer with a monomer comprising a first reactive group.

The use of an adherent polymer provides various advantages for forming a biomolecule-presenting coating. For example, the coating can easily be formed on substrates that have hydrophobic surfaces. Another advantage is that a stable, durable, and compliant coating can be formed on substrates without the need for covalent bonding between the polymeric material and the substrate. This can be particularly useful when the substrate has few or no moieties on its surface which can be used for covalent bonding. Yet another advantage is that a bioactive agent compatible with the acrylate polymer can be optionally included in the coating, which can be useful when the coating is formed on the surface of an implantable medical device. In the case where a bioactive agent is optionally included in an implantable medical device coating, it can be released from the coating to provide a local therapeutic effect in vivo.

In a related aspect a biomolecule-containing coating is prepared by mixing an adherent polymer with a second polymeric material having a first reactive group. The adherent polymer is able to form a coating on the surface of the device in which the second polymeric material can become immobilized. The second polymeric material can be reacted with a biomolecule having a second reactive group to couple to the biomolecule to the second polymeric material, and thus immobilizing the biomolecule on the surface of the device. Therefore, in some aspects, the invention provides a coating comprising (a) an acrylate polymer, (b) a second polymer that is different than the acrylate polymer, the second polymer comprising a plurality of first reactive groups, and (c) a biomolecule coupled to the second polymer via the first reactive group. In one aspect the second polymeric material is a non-film forming polymer. For example, preferred second polymeric materials include poly(carbodiimide). Other suitable second polymers include a plurality of pendent amine groups. In another preferred aspect, the ratio of the first polymeric material to the second polymeric material is in the range of about 1:10 to about 10:1, and more specifically about 3:7 to about 7:3.

In another aspect, the coating includes a coated layer that includes a polymeric material having a photoreactive group and a first reactive group, and a second layer that includes a biomolecule having a second reactive group. In the coating, the photoreactive group has been activated and binds the polymeric material to the surface of the article, and the first reactive group is reacted with the second reactive group to couple the biomolecule to the polymeric material.

For example, in some aspects, the polymeric material comprises a pendant amine group and a pendent photoreactive group. Preferably a plurality of amine groups are pendent along the length of the polymer, to provide a polymeric layer having a high density of amine groups. The amine group can be reacted with a second reactive group included on the biomolecule that is an amine reactive group. Polymeric material comprising pendant amine and photogroups can be selected from the group of polylysine, polyomithine, polyethylenimine, polypropylenimine, and polyamidoamine.

Any photoreactive group can be used that allows the polymer to become bound to the surface. For example, the photoreactive group can be selected from photoreactive aryl ketones, such as acetophenone, benzophenone, anthraquinone, anthrone, and anthrone-like heterocycles (for example, heterocyclic analogs of anthrone such as those having nitrogen, oxygen, or sulfur in the 10-position), or their substituted (for example, ring substituted) derivatives.

Therefore, in another aspect, a coating can be formed by a method that comprising the steps of (a) disposing a polymeric material on a surface, the polymeric material comprising (i) a pendent photoreactive group and (ii) a first reactive group that is an amine group; (b) disposing a biomolecule comprising a second reactive group that is an amine-reactive group; and (c) treating the polymeric material to activate the photoreactive group to bind the polymeric material to the surface. The step of treating can be performed before, during, or after the biomolecule is disposed on the surface.

In other aspects, the invention provides a coating with at least three layers: a layer including the polymeric material having the photogroup and first reactive group, a layer including a anionic polymer, and a layer including a biomolecule having a second reactive group.

Therefore, in some aspects, the method can include an additional step of disposing an anionic polymer. For example, the method of the invention can include steps of (a) disposing a polymeric material on a surface, the polymeric material comprising (i) a photoreactive group and (ii) a first reactive group; (b) disposing an anionic polymer, (c) disposing a biomolecule comprising a second reactive group, the second reactive group reactive with the first reactive group; and (d) treating the polymeric material to activate the photoreactive group to bind the polymeric material to the surface. Steps (a) and/or (b) can be repeated, as desired. The anionic polymer can be a sulfated polysaccharide such as dextran sulfate.

### DETAILED DESCRIPTION

The embodiments of the present invention described below are not intended to be exhaustive or to limit the invention to the precise forms disclosed in the following detailed description. Rather, the embodiments are chosen and described so that others skilled in the art can appreciate and understand the principles and practices of the present invention.

All publications and patents mentioned herein are hereby incorporated by reference. The publications and patents disclosed herein are provided solely for their disclosure. Nothing herein is to be construed as an admission that the inventors are not entitled to antedate any publication and/or patent, including any publication and/or patent cited herein.

The present invention provides reagents and methods for providing a coating to the surface of an article, the coating including a polymeric material and a biomolecule coupled to the polymeric material. The coating can be formed on a variety of articles, wherein it is desired to have the functional properties of a biomolecule on the surface. The coatings of the present invention can be formed on articles used in various technologies, including, but not limited to, articles that are used in medical technologies including implantable medical devices, surgical equipment, and surgical instruments; assay instrumentation and products, such as biosensor-based systems, chemiluminescence detection systems, immunoassay systems; assay plates, including 1536, 384, and 96 well plates; solid supports; microbiology equipment such as fermentation equipment and bacteriological testing equipment; tubing; cell biology articles, such as cell assay kits; cell biology equipment, such as tissue processing articles, flow cytometry articles, and screening articles; cell culture articles such as culture jars, cell collection systems, cell harvesters, cell separation articles, culture dishes, culture flasks, culture plates, culture roller bottles, culture slides, and culture tubes; bioreactors; fermentors; hollow fiber systems; perfusion systems; suspension systems; chromatography and separation systems, such as affinity columns and biomolecular columns; detectors, such as amperometric detectors, chemiluminescence detectors, electrochemical detectors, fluorescence detectors, and MALDI-TOF mass spec; drug discovery systems, such as articles used in high throughput systems; filters and filtration equipment, including bacteriological filters, glass fibers, affinity membranes, microbial membranes, microfilters, tissue culture; genomic and proteomic system articles, such as microarray articles including slides, chips, and microfluidic articles; immunochemical systems including ELISA and immunoassay kits; microscope slides and accessories; nucleic acid equipment including automated sequencers; nucleic acid analysis kits; protein analysis equipment; ampules; glassware; petri dishes; test tubes; vials; and plastic and micro pipets.

The coatings can be formed on a wide variety of materials used to fabricate the article or device. The materials to form the structure of the article are referred to herein as “article materials” or “device materials” whereas the materials used to form the polymeric coatings herein referred to as “coating materials.” In many cases, the article can be formed from one or more biomaterial(s) if the coated article is to be placed in contact with a biological fluid or tissue (such as being implanted in the body).

Example of materials which can be used to form the article onto which the coating can be formed include synthetic polymers, including oligomers, homopolymers, and copolymers resulting from either addition or condensation polymerizations. Examples of suitable addition polymers include, but are not limited to, acrylics such as those polymerized from methyl acrylate, methyl methacrylate, hydroxyethyl methacrylate, hydroxyethyl acrylate, acrylic acid, methacrylic acid, glyceryl acrylate, glyceryl methacrylate, methacrylamide, and acrylamide; vinyls such as ethylene, propylene, vinyl chloride, vinyl acetate, vinyl pyrrolidone, and vinylidene difluoride.

Examples of condensation polymers include, but are not limited to, nylons such as polycaprolactam, polylauryl lactam, polyhexamethylene adipamide, and polyhexamethylene dodecanediamide, and also polyurethanes, polycarbonates, polyamides, polysulfones, poly(ethylene terephthalate), polylactic acid, polyglycolic acid, polydimethylsiloxanes, and polyetherketone.

Other natural materials can be used to form the article, including human tissue such as bone, cartilage, skin and teeth; and other organic materials such as wood, cellulose, compressed carbon, and rubber. Other suitable materials include metals and ceramics. The metals include, but are not limited to, titanium, Nitinol, stainless steel, tantalum, and cobalt chromium. A second class of metals includes the noble metals such as gold, silver, copper, and platinum uridium. Alloys of metals are suitable for biomaterials as well. The ceramics include, but are not limited to, silicon nitride, silicon carbide, zirconia, and alumina, as well as glass, silica, and sapphire.

Combinations of ceramics and metals are another class of materials. Another class of biomaterials is fibrous or porous in nature. The surface of such biomaterials can be pretreated (for example, with a Parylene-containing coating composition) in order to alter the surface properties of the biomaterial, when desired.

The materials can be used to fabricate a variety of implantable medical devices. The medical device can be any device that is introduced temporarily or permanently into a mammal for the prophylaxis or treatment of a medical condition. These devices include any that are introduced subcutaneously, percutaneously or surgically to rest within an organ, tissue, or lumen of an organ, such as arteries, veins, ventricles, or atria of the heart.

Compositions of this invention can be used to coat the surface of a variety of implantable medical devices. In some aspects the coatings that are formed provide a biocompatible surface to the implantable medical device. The biocompatible surface can enhances the ability of the medical device to function or exist in contact with biological fluid and/or tissue of a living organism with a net beneficial effect on the living organism.

The inventive coatings can be formed on devices such as drug-delivering vascular stents; other vascular devices (e.g., grafts, catheters, valves, artificial hearts, heart assist devices, ventricular assist devices); implantable defibrillators; blood oxygenator devices; surgical devices; tissue-related materials; membranes; shunts for hydrocephalus; wound management devices; endoscopic devices; infection control devices; orthopedic devices; dental devices, urological devices; colostomy bag attachment devices; ophthalmic devices; glaucoma drain shunts; synthetic prostheses; intraocular lenses; respiratory, peripheral cardiovascular, spinal, neurological, dental, and ear/nose/throat devices (e.g., ear drainage tubes); renal devices; and dialysis (e.g., tubing, membranes, grafts).

The inventive coatings can be formed on other devices such self-expanding stents (e.g., made from nitinol), balloon-expanded stents (e.g., prepared from stainless steel), degradable coronary stents, non-degradable coronary stents, peripheral coronary stents, endovascular stents, intraaortic baloons, urinary catheters (e.g., surface-coated with antimicrobial agents), penile implants, sphincter devices, urethral devices, bladder devices, renal devices, vascular implants and grafts, intravenous catheters (e.g., treated with antithrombotic agents), small diameter grafts, artificial lung catheters, electrophysiology catheters, pacemaker leads, anastomosis devices, vertebral disks, bone pins, suture anchors, hemostatic barriers, clamps, surgical staples/sutures/screws/plates/clips, atrial septal defect closures, electro-stimulation leads for cardiac rhythm management (e.g., pacer leads), glucose sensors (long-term and short-term), blood pressure and stent graft catheters, blood oxygenator tubing, blood oxygenator membranes, blood bags, birth control devices, breast implants; benign prostatic hyperplasia and prostate cancer implants, bone repair/augmentation devices, breast implants, cartilage repair devices, orthopedic joint implants, orthopedic fracture repairs, tissue adhesives, tissue sealants, tissue scaffolds, CSF shunts, dental implants, dental fracture repair devices, implanted drug infusion tubes, intravitreal drug delivery devices, nerve regeneration conduits, oncological implants, electrostimulation leads, pain management implants, spinal/orthopedic repair devices, surgical blood salvage disposal sets, wound dressings, embolic protection filters, abdominal aortic aneurysm grafts, heart valves (e.g., mechanical, polymeric, tissue, percutaneous, carbon, sewing cuff), valve annuloplasty devices, mitral valve repair devices, vascular intervention devices, left ventricle assist devices, neuro aneurysm treatment coils, neurological catheters, left atrial appendage filters, central venous access catheters, hemodialysis devices, hemodialysis catheters, catheter cuff, anastomotic closures, vascular access catheters, cardiac sensors, intravascular sensors, uterine bleeding patches, urological catheters/stents/implants, in vitro diagnostics, aneurysm exclusion devices, neuropatches, Vena cava filters, urinary dialators, endoscopic surgical tissue extractors, atherectomy catheters, clot extraction catheters, PTA catheters, PTCA catheters, stylets (vascular and non-vascular), coronary guidewires, drug infusion catheters, esophageal stents, circulatory support systems, angiographic catheters, transition sheaths and dialators, coronary and peripheral guidewires, hemodialysis catheters, neurovascular balloon catheters, tympanostomy vent tubes, cerebro-spinal fluid shunts, defibrillator leads, percutaneous closure devices, drainage tubes, thoracic cavity suction drainage catheters, electrophysiology catheters, stroke therapy catheters, abscess drainage catheters, biliary drainage products, dialysis catheters, central venous access catheters, and parental feeding catheters.

The methods and compositions described herein are particularly useful for those devices that will come in contact with aqueous systems, such as bodily fluids. In some aspects of the invention, a biocompatible agent coupled to the polymeric material of the coating to provide a biocompatible surface to a medical device.

While the coating of the present invention includes a biomolecule coupled to polymeric material via a reactive group, the coating can also include other optional materials. More specifically, the coating can include other optional coated layers. As used herein, the term “layer” or “coated layer” will refer to a layer of one or more coated materials of sufficient dimensions (for example, thickness and area) for its intended use over the entire, or less than the entire, portion of an article surface. A “coating” as described herein can include one or more “coated layers,” each coated layer including one or more coating components.

One or more additional optional coated layers can be included in the coating on the article. Generally, if one or more additional optional coated layers are present in the coating, the additional layer(s) are located between the polymeric coating having the first reactive group and the surface of the device. Therefore, when referring to the step of providing the polymeric coating having the first reactive group to a surface, the surface may be that of the device itself, or the surface of the device with the additional optional coated layers.

For example, the polymeric material including the first reactive group can be disposed on a medical device pre-coated with a Parylene™ or a Parylene™ derivative. “Parylene” is both a generic name for a known group of polymers based on p-xylylene and made by vapor phase polymerization, and a name for the unsubstituted form of the polymer; the latter usage is employed herein. Parylene™ or a Parylene™ derivative is created by first heating p-xylylene or a suitable derivative at an appropriate temperature (for example, at about 100-150° C.) to produce the cyclic dimer di-p-xylylene (or a derivative thereof). The resultant solid can be separated in pure form, and then cracked and pyrolyzed at an appropriate temperature (for example, at about 690° C.) to produce a monomer vapor of p-xylylene (or derivative); the monomer vapor is cooled to a suitable temperature (for example, below 30° C.) and allowed to condense on the desired object, for example, on the surface of the medical device.

As indicated, Parylene™ and Parylene™ derivative precoatings applicable by vapor deposition are known for a variety of biomedical uses, and are commercially available from or through a variety of sources, including Specialty Coating Systems (100 Deposition Drive, Clear Lake, Wis. 54005), Para Tech Coating, Inc. (35 Argonaut, Aliso Viejo, Calif. 92656) and Advanced Surface Technology, Inc. (9 Linnel Circle, Billerica, Mass. 01821-3902).

The polymeric material having the first reactive group can also be disposed on a medical device precoated with a silane compound. Suitable silane compound precoatings are described in U.S. Pat. No. 6,706,408.

These types of optional base coated layers can be particularly useful for providing a surface that can be reacted with a photoreactive group pendent from the polymeric material having a first reactive group. In cases where the article material does not provide a source of abstractable hydrogens, and wherein it is desired to utilize a polymeric material having a pendent photoreactive group, providing a base coat is desired.

According to the invention, a polymeric material is disposed or provided to the surface of an article that includes a first reactive group to form a coated layer. A biomolecule including a second reactive group, when disposed on the coated layer, becomes coupled to the polymeric material via the first reactive group.

The polymeric material preferably includes polymers that are biologically stable and that can be organic or inorganic, or synthetic or naturally occurring substances. The polymeric material can include one or more polymers that are selected from a wide variety of polymers.

In some embodiments, the polymeric material includes an adherent polymer with a first reactive group. In these embodiments a pendent photoreactive group is not necessarily required for the formation of a coated layer on a surface. That is, the adherent polymer can be deposited on a surface and covalent bonding between the adherent polymer and the surface is not required for the formation of a suitable coating.

In some aspects of the invention, the adherent polymer is an acrylate polymer having pendent first reactive groups. As referred to herein, an “acrylate polymer” includes both acrylate copolymers and acrylate homopolymers.

Acrylate polymers having first reactive groups can be formed by various different synthetic processes. In one preferred process, the acrylate polymer having first reactive groups is formed by the polymerization of (a) acrylate monomers and (b) monomers having first reactive groups. For example, an alkyl(meth)acrylate or aromatic(meth)acrylate monomer can be copolymerized with a monomer having an amine-reactive group.

Alternatively, the acrylate polymer can be formed by copolymerizing an acrylate monomer with a monomer having a primary amine group, wherein the amine group represents the first reactive group.

In yet another process, the acrylate polymer having pendent first reactive groups can be formed by the reaction of an acrylate polymer with a compound that provides a first reactive group.

Suitable acrylate polymers can be selected from (alkyl(meth)acrylate) polymers and (aromatic(meth)acrylate) polymers, where “(meth)” will be understood by those skilled in the art to include such molecules in either the acrylic and/or methacrylic form (corresponding to the acrylates and/or methacrylates, respectively).

Examples of suitable (alkyl(meth)acrylate) polymers include those with alkyl chain lengths from 2 to 8 carbons, inclusive, and with molecular weights from 50 kilodaltons to 900 kilodaltons. In one preferred embodiment the polymeric material includes a poly(alkyl (meth)acrylate) with a molecular weight of from about 100 kilodaltons to about 1000 kilodaltons, preferably from about 150 kilodaltons to about 500 kilodaltons, most preferably from about 200 kilodaltons to about 400 kilodaltons. An example of a particularly preferred is polymer is a (n-butyl methacrylate) polymer. Examples of other preferred polymers include (n-butyl methacrylate-co-methyl methacrylate) polymers, poly(n-butyl methacrylate-co-isobutyl methacrylate) polymers, and poly(t-butyl methacrylate) polymers.

Examples of suitable (aromatic(meth)acrylate) polymers include (aryl(meth)acrylate) polymers, (aralkyl(meth)acrylate) polymers, (alkaryl(meth)acrylate) polymers, (aryloxyalkyl(meth)acrylate) polymers, and (alkoxyaryl(meth)acrylate) polymers.

Examples of suitable (aryl(meth)acrylate) polymers include (9-anthracenyl methacrylate) polymers, (chlorophenyl acrylate) polymers, (methacryloxy-2-hydroxybenzophenone) polymers, (methacryloxybenzotriazole) polymers, (naphthyl acrylate) polymers, (naphthylmethacrylate) polymers, 4-nitrophenylacrylate polymers, (pentachloro(bromo, fluoro) acrylate) and methacrylate polymers, (phenyl acrylate) polymers, and (phenyl methacrylate) polymers. Examples of suitable (aralkyl(meth)acrylate) polymers include (benzyl acrylate) polymers, (benzyl methacrylate) polymers, (2-phenethyl acrylate) polymers, (2-phenethyl methacrylate) polymers, and (1-pyrenylmethyl methacrylate) polymers. Examples of suitable (alkaryl(meth)acrylate) polymers include (4-sec-butylphenyl methacrylate) polymers, (3-ethylphenyl acrylate) polymers, and (2-methyl-1-naphthyl methacrylate) polymers. Examples of suitable (aryloxyalkyl(meth)acrylate) polymers include (phenoxyethyl acrylate) polymers, (phenoxyethyl methacrylate) polymers, and (polyethylene glycol phenyl ether acrylate) and (polyethylene glycol phenyl ether methacrylate) polymers with varying polyethylene glycol molecular weights. Examples of suitable (alkoxyaryl(meth)acrylate) polymers include (4-methoxyphenyl methacrylate) polymers, (2-ethoxyphenyl acrylate) polymers, and (2-methoxynaphthyl acrylate) polymers.

Acrylate or methacrylate monomers or polymers and/or their parent alcohols are commercially available from Sigma-Aldrich (Milwaukee, Wis.) or from Polysciences, Inc, (Warrington, Pa.).

Other useful polymers and mixtures of polymers that can be included in the coating composition are described in commonly assigned U.S. Provisional Patent Application entitled, “COATING COMPOSITIONS FOR BIOACTIVE AGENTS,” having attorney docket number 9896.166.1.

In these embodiments a pendent photoreactive group is not necessarily required for the formation of a coated layer on a surface. That is, the adherent polymer can be deposited on a surface and covalent bonding between the adherent polymer and the surface is not required for the formation of a suitable coating.

In other embodiments, the coating includes an adherent polymer in mixture with a second polymeric material having a first reactive group. The first polymeric material is able to form a coating on the surface of the device in which the second polymeric material can become immobilized. The second polymeric material can be reacted with a biomolecule having a second reactive group to couple to the biomolecule to the second polymeric material, thus immobilizing the biomolecule on the surface of the device. In a preferred aspect, the first polymeric material is an acrylate polymer, and the second polymer is a polymer that is different than the acrylate polymer, but able to be mixed with the acrylate polymer, and which also includes a plurality of first reactive groups. Preferred second polymeric materials include, for example, poly(carbodiimide) and polymers such as polylysine, polyomithine, polyethylenimine, polypropylenimine, and polyamidoamine.

It is desirable to use a reactive pair that allows efficient and rapid coupling of the polymeric material to the biomolecule. For example, it is desirable to use first and second reactive groups that are able to react with each other and form a bond under conditions that are not detrimental to either the polymeric material or the biomolecule. Preferred first and second reactive groups can be under suitable conditions to allow for chemical reaction and bond formation.

Contemplated reactive pairs include, but are not limited to, the reactive pairs as set forth in Table 1. Table 1 lists reactive pairs having reactive group A that is reactive with corresponding reactive group B. If the first reactive group that is pendent from the polymeric material can be selected from reactive group A of Table 1, the second reactive group pendent from the biomolecule is selected from reactive group B; accordingly if the second reactive group that is pendent from the biomolecule can be selected from reactive group A of Table 1, the first reactive group pendent from the polymeric material is selected from reactive group B.

TABLE 1

| Reactive group A | Reactive group B |
|---|---|
| amine, hydroxyl, sulfhydryl | N-oxysuccinimide (“NOS”) |
| amine | Aldehyde |
| amine | Isothiocyanate |
| amine, sulfhydryl | Bromoacetyl |
| amine, sulfhydryl | Chloroacetyl |
| amine, sulfhydryl | Iodoacetyl |
| amine, hydroxyl | Anhydride |
| aldehyde | Hydrazide |
| amine, hydroxyl, carboxylic acid | Isocyanate |
| amine, sulfhydryl | Maleimide |
| sulfhydryl | Vinylsulfone |

Amine also includes hydrazide (R-NH—NH₂)

The reaction between the first and second reactive groups generally takes place when the biomolecule is brought into contact with, or is mixed with, the polymeric material. For example, the reaction can take place when the biocompatible agent is disposed on the polymeric material. In most cases, the reaction between reactive group A and B is sufficient under ambient conditions to form a covalent bond. Adjustments in the reaction conditions, for example, the temperature and pH, can be performed to improve reaction efficiency and/or control how the reaction takes place.

In some embodiments wherein an amine is used as a reactive group the polymeric material can be linked to the biomolecule through, for example, an amine, amide, imine, thiourea, or urea bond.

One or more first reactive groups, or combinations of different first reactive groups, can be added to a polymer of the polymeric material. A number of different approaches can be used to prepare a polymer having one or more first reactive groups. For example, a polymer having a first reactive group can be prepared by obtaining or synthesizing a monomer having a first reactive group. The monomer having a first reactive group can be polymerized or copolymerized with other monomers to form the polymer having the first reactive group.

The first reactive group is present on the polymeric material in an amount sufficient to promote the coupling of the biomolecule to the polymeric material. In some aspects, the greater than about 5% of the monomeric units of the polymeric material include a first reactive group. In other aspects about 5% to about 20% of the monomeric units of the polymeric material include a first reactive group. In yet other aspects about 20% or less of the monomers of the polymeric material include first reactive groups.

In other aspects, a polymeric material can be obtained that includes one or more first reactive groups and there is not a need to derivatize the polymeric material to add a first reactive group. For example, a polymer can be chosen that provides, for example, pendent amine, hydroxyl, sulfhydryl, hydroxyl, or carboxylic acid groups, or combinations thereof.

The reactive pair allows the polymeric material to be coupled to the biomolecule in a variety of ways, as illustrated herein. For example, in some aspects, a polymer having pendent hydroxyl groups (as a first reactive group) can be reacted with a biomolecule having pendent isocyanate or anhydride groups (the second reactive group). In these cases, there may not be a need to further derivatize the polymer to provide a first reactive group.

In other cases, the polymer can be derivatized to add one or more first reactive groups. For example, amino groups can also be added by the reaction of acid polymers with aziridines such as ethylene imine, or by the reaction of epoxy and blocked ketimines; other techniques that are known in the art for adding amine functionality to polymers can be carried out as desired.

In some embodiments, the polymeric material includes a polymer having a photoreactive group and first reactive group. The use of a polymer having a photoreactive group can allow the polymer, which provides a base material for the immobilization of the biomolecule, to be bound at particular locations on the surface. For example, a polymeric material having a photogroup can be deposited on a surface and the surface can be treated with irradiation at one or more particular locations on the surface to bind the polymeric material to those locations. The biomolecule can then be disposed on the surface which will bind to the polymeric material through reaction of the first and second reactive groups, leaving the biocompatible material linked to the surface at the locations that were treated with irradiation.

In preferred embodiments, in addition to the photoreactive group, the polymeric material includes a plurality of pendent amine groups reactive with the second reactive group on the biomolecule. The reactive amine groups are preferably primary or secondary amines that can be pendent along the length of the polymer in a random or ordered fashion. The amine groups can be pendent from a homopolymer or a copolymer. Preferably, the amine-containing polymer provides a polymeric coated layer that has a high density of amine groups. This advantageously can allow for an increased amount of the biomolecule coupled to the surface, and therefore improved biomolecule-associated properties.

In some preferred embodiments, the polymer is selected from the group consisting of polylysine, polyornithine, polyethylenimine, polypropylenimine, and polyamidoamine. In one preferred embodiment the polymeric material includes polyethyeneimine. Polymers having pendent amine groups can be readily derivatized with a photoreactive group by reacting a portion of the pendent amine groups with a photo-reactive moiety that is reactive with an amine group, such as 4-benzoylbenzoyl chloride.

In other embodiments, polymerization is performed to provide a polymer having pendent amine groups and pendent photoreactive groups. For example, primary amine containing monomers such as N-(2-amino-2-methylpropyl)methacrylamide, 2-aminoethyl methacrylate (AEMA), p-aminostyrene, N-(2-aminoethyl)methacrylamide, N-(3-aminopropyl) methacrylamide, allyl amine, or combinations thereof can be copolymerized with a monomer having a pendent photoreactive group to provide a polymer having pendent amine groups and photogroups. These amine-containing monomers can also be copolymerized with other non-primary amine-containing monomers, such as acrylamide, methacrylamide, vinyl pyrrolidinone, or derivatives thereof, to provide a polymer having desired properties, such as a desired density of amine groups and photoreactive groups.

Other suitable polymers that have reactive amine groups include polymers that are formed from monomers such as 2-aminomethylmethacrylate, 3-(aminopropyl)-methacrylamide, and diallylamine. Dendrimers that include photogroups and pendent amine groups can also be used.

In one embodiment of the invention, the polymeric material includes a polymer having a photogroup and a first reactive group that is an amine, and the biomolecule includes an amine-reactive group. In one preferred embodiment, the amine reactive group can be selected from the group consisting of NOS, aldehyde, isothiocyanate, isocyanate, bromoacetyl, chloroacetyl, iodoacetyl, maleimide. In particular embodiments the biocompatible agent is heparin having an amine-reactive group, for example, aldehyde or isothiocyanate.

The photoreactive group that is present on the polymer allows for bonding of the polymer to a surface. Photoreactive groups respond to a specific applied external ultraviolet or visible light source to undergo active specie generation with resultant covalent bonding to an adjacent chemical structure, for example, as provided by the same or a different molecule. These groups retain their covalent bonds unchanged under conditions of storage but that, upon activation by a light source, form covalent bonds with other molecules. Photoreactive groups can generate active species such as free radicals and particularly nitrenes, carbenes, and excited states of ketones, upon absorption of electromagnetic energy.

Reactive aryl ketones are preferred photoreactive groups. Aryl ketone photoreactive groups include acetophenone, benzophenone, anthraquinone, anthrone, and anthrone-like heterocycles (for example, heterocyclic analogs of anthrone such as those having nitrogen, oxygen, or sulfur in the 10-position), or their substituted (for example, ring substituted) derivatives. Examples of preferred aryl ketones include heterocyclic derivatives of anthrone, including acridone, xanthone, and thioxanthone, and their ring substituted derivatives. Particularly preferred are thioxanthone, and its derivatives, having excitation energies greater than about 360 nm.

The functional groups of such ketones are preferred since they are readily capable of undergoing the activation/inactivation/reactivation cycle described herein. Benzophenone is a particularly preferred latent reactive moiety, since it is capable of photochemical excitation with the initial formation of an excited singlet state that undergoes intersystem crossing to the triplet state. The excited triplet state can insert into carbon-hydrogen bonds by abstraction of a hydrogen atom (from a support surface, for example), thus creating a radical pair. Subsequent collapse of the radical pair leads to formation of a new carbon-carbon bond. If a reactive bond (for example, carbon-hydrogen) is not available for bonding, the ultraviolet light-induced excitation of the benzophenone group is reversible and the molecule returns to ground state energy level upon removal of the energy source. Photoactivatible aryl ketones such as benzophenone and acetophenone are of particular importance inasmuch as these groups are subject to multiple reactivation in water and hence provide increased coating efficiency.

Preparation of polymeric material having pendent photoreactive groups can be achieved by a variety of different methods. For example, a polymer having pendent photoreactive groups can be first prepared by preparing a copolymer and then reacting the copolymer with compounds that lead to the photoderivatization of the copolymer.

In some cases, an article having a first coated layer can be provided to a user, who then can dispose a desired biomolecule on the first coated layer to couple it to the polymeric material to form the coating. That is, an article having a first coated layer that includes a polymeric material having a first reactive group can be pre-prepared. The article can then later be obtained by a user who disposes a biomolecule on the coated layer in order to form a coating that includes the biomolecule coupled to a polymeric material. The polymeric material of the coated layer can be an adherent polymer, such as the acrylate polymers having first reactive groups, or a polymer having pendent amine and photoreactive groups.

This arrangement may allow improved control over the formation of the coating, particularly if the user possesses techniques or equipment that allows for optimal formation of the biomolecule on the coated layer, and/or the provider possesses techniques or equipment that allows for optimal formation of the coated layer.

Therefore, in some aspects, the invention includes the methods of (a) obtaining an article having a coated layer, the coated layer comprising a polymeric material comprising an acrylate polymer having a first reactive group, and (b) disposing a biomolecule comprising a second reactive group on the coating, wherein the step of disposing couples the biomolecule to the polymeric material of the coated layer. In other aspects the coated layer comprises a polymeric material comprising a photoreactive group and a plurality of amine groups, wherein the photoreactive group couples the amine group to the article.

Other polymers that can serve as a backbone for the polymer having a first reactive group and a photoreactive group include, but are not limited to, polyacrylamides, polymethacrylamides, polyvinylpyrrolidone, polyacrylic acid, polyethylene glycol, polyvinyl alcohol, poly(HEMA), and copolymers thereof. Particularly useful polymers include monomers selected from acrylamide, methacrylamide, vinyl pyrrolidinone, or derivatives thereof; include a reactive group selected from NOS, aldehyde, isothiocyanate, isocyanate, bromoacetyl, chloroacetyl, iodoacetyl, and maleimide; and include a photoreactive group.

In some embodiments, the coating also includes an anionic polymer. For example, the coating can include one or more layers of an anionic polymer such as dextran sulfate. The anionic polymer can be included in coatings that include polymeric material having either (a) an adherent polymer, such as an acrylate polymer having a first reactive group, or (b) a polymer having a photoreactive group and a plurality of amine groups.

The term “biomolecule” is used in its broadest sense and refers to any type of component that can be coupled to the polymeric coating on the surface of the coated article, wherein the component exerts a biological effect, or has any sort of biologically-based function. Examples of biologically-based functions include ligand or analyte binding, as provided by, for example, antibodies, nucleic acids, or proteins that serve as receptors. Other examples of biologically-based functions include enzymatic catalysis, as provided by, for example, protein enzymes or nucleic acid enzymes. Examples of components that exert biological effects include polysaccharides, peptides, proteins, or small natural or synthetic molecules that can have a direct or indirect effect on, for example, a cell or other component in vivo or in vitro.

The biomolecule is coupled to the polymeric material of the coating via the reactive pair, wherein a first reactive group present on the polymeric material is reacted with a second reactive group present on the biomolecule. The second reactive group on the biomolecule may be intrinsic to the biomolecule, meaning that no derivation or modification of the biomolecule is necessary to provide the second reactive group. For example, amine groups are naturally present on many proteins and polysaccharides, and can be suitable as the second reactive group. Alternatively, the biomolecule can be modified to provide a second reactive group.

For example, a biomolecule having an amine-reactive group can be prepared by a number of techniques. A bifunctional reagent having two similar or different amine-reactive groups, such as NHS esters, sulfo-NHS esters, malemides, or imido esters, can be combined with a biomolecule having amine groups and amenable towards modification with one of these reagents to provide a biomolecule with amine-reactive groups. If the biomolecule is a polymeric molecule, approaches can be taken to provide the amine reactive group at a particular location the polymer for example at the polymer ends or along the length of the polymer. Heparin having amine reactive groups may be prepared by a number of different approaches. For example, heparin having amine-reactive aldehyde groups can be prepared by treating heparin with nitric acid. Another approach is to modify heparin with dicyclohexylcarbodiamide and carbon disulfide in a non-aqueous solvent and at low pH to provide pendent isothiocyanate reactive groups on the heparin.

Essentially any biomolecule can be attached to the a target surface via the polymeric coating of the present invention. In a preferred aspect, the biomolecule is a naturally occurring polymer or derivative thereof. In another preferred aspect, the biomolecule is selected from the group of polypeptides, polysaccharides, and polynucleotides. In yet another preferred aspect, the biomolecule is a polymucosaccharide.

In some aspects, the biomolecule is a biocompatible agent that can improve the biocompatibility of the medical device, including those medical devices having a variety of biomaterial surfaces as described herein. Accordingly, the biocompatible agent has at least the properties of providing biocompatibility and being able to be coupled to the polymeric material. The polymeric material allows the biocompatible agent to be stably presented on the surface of the coated article.

In preferred embodiments, the biocompatible agent, when coupled to the medical device surface, can serve to shield the blood from the underlying medical device material. Suitable biocompatible agents preferably reduce the likelihood for blood components to adhere to the medical device and activate, thus reducing the formation of thrombus or emboli (blood clots that release and travel downstream). The biocompatible surface thus enhances the ability of the medical device to function or exist in contact with biological fluid and/or tissue of a living organism with a net beneficial effect on the living organism. The biocompatible surface can provide one or more advantages, such as increased patient safety, improved device performance, reduced adherence of unwanted blood components, inhibition of blood clotting, maintenance of device surfaces free of cellular debris, and/or extension of the useable lifetime of the device.

Any suitable implantable medical device, such as a stent or a synthetic graft having a structure adapted for the introduction into a patient, can be provided with a biocompatible coating. In some embodiments the device is coated with coating composition that includes one or more bioactive agents for delivery of a drug or pharmaceutical substance to tissues adjacent the site of implantation. In some embodiments the device can be a drug-eluting stent having a biocompatible surface. The methods and compositions of the invention in connection with drug-eluting stent can be particularly useful because these devices are designed to reside in the body for extended periods of time, thus increasing risk of adverse body reactions to the device.

One preferred biocompatible agent is heparin. Heparin, as used herein, is meant to encompass all forms and preparations of heparin including, but not limited to, sodium heparin, low molecular weight heparin, high affinity heparin, low affinity heparin, modified heparin, and treated heparin (such as oxidized heparin). According to the invention the heparin component also includes a reactive group, or more than one reactive group, that is pendent from any portion of the heparin molecule and reactive with a first reactive group that is pendent from the polymer. In some cases, the group that is reactive on the heparin molecule can be introduced when heparin is treated. For example, heparin can be treated to introduce a group on one terminus (that is, a second reactive group) that is reactive with the first reactive group on the polymer, which includes the first reactive group and the photogroup. For example, heparin can be treated with nitrous acid, causing partial depolymerization and introduction of aldehyde groups on its ends. In other aspects, the reactive group is provided by the heparin itself, for example, the naturally-occurring amine groups pendent from the heparin serve as the second reactive group. In these aspects it is not required that heparin is modified to include a second reactive group.

Therefore, in other aspects, the biocompatible material comprises a second reactive group that is an aldehyde and the polymeric material includes a polymer having a first reactive group that is aldehyde reactive. For example, the polymer can include an amine group or a hydrazide group.

Some preferred coatings include an acrylate polymer, the polymer adhered to the surface of the medical device and heparin coupled to the acrylate polymer via an amine-reactive group that is pendent from the heparin.

Another preferred coating includes a polymer having pendent amine groups, the polymer bound to the surface of the medical device via a photogroup and heparin coupled to a pendent amine group of the polymer via an amine reactive group that is pendent from the heparin.

Other biocompatible agents can be coupled to the polymeric material to provide antirestenotic effects, such as antiproliferative, anti-platelet, and/or antithrombotic effects.

Representative examples of other biocompatible agents having antithrombotic effects (thrombin inhibitors) include hirudin, lysine, prostaglandins, argatroban, forskolin, vapiprost, prostacyclin and prostacyclin analogs, D-ph-pr-arg-chloromethylketone (synthetic antithrombin), dipyridamole, glycoprotein IIb/IIla platelet membrane receptor antibody, coprotein IIb/IIIa platelet membrane receptor antibody, recombinant hirudin, thrombin inhibitor (such as commercially available from Biogen), chondroitin sulfate, modified dextran, albumin, streptokinase, tissue plasminogen activator (TPA), urokinase, nitric oxide inhibitors, and the like.

The biocompatible agent can also be an inhibitor of the GPIIb-IIIa platelet receptor complex, which mediates platelet aggregation. GPIIb/IIIa inhibitors can include monoclonal antibody Fab fragment c7E3, also know as abciximab (ReoPrO™), and synthetic peptides or peptidomimetics such as eptifibatide (Integrilin™) or tirofiban (Agrastat™).

In some embodiments, the biocompatible agent can include anti-inflammatory agents, immunosuppressive agents, cell attachment factors, receptors, ligands, growth factors, antibiotics, enzymes, nucleic acids, and the like. Biocompatible agents having antiproliferative effects include, for example, actinomycin D, angiopeptin, c-myc antisense, paclitaxel, taxane, and the like. Examples of immunosuppressive agents include cyclosporine, CD-34 antibody, everolimus, mycophenolic acid, sirolimus, tacrolimus, and the like.

Additionally, the biocompatible agent can include surface adhesion molecules or cell-cell adhesion molecules. Exemplary cell adhesion molecules or attachment proteins (such as extracellular matrix proteins including fibronectin, laminin, collagen, elastin, vitronectin, tenascin, fibrinogen, thrombospondin, osteopontin, von Willibrand Factor, bone sialoprotein and active domains thereof), or a hydrophilic polymer such as hyaluronic acid, chitosan or methyl cellulose, and other proteins, carbohydrates, and fatty acids. Exemplary cell-cell adhesion molecules include N-cadherin and P-cadherin and active domains thereof.

Exemplary growth factors include fibroblastic growth factors, epidermal growth factor, platelet-derived growth factors, transforming growth factors, vascular endothelial growth factor, bone morphogenic proteins and other bone growth factors, and neural growth factors.

Exemplary ligands or receptors include antibodies, antigens, avidin, streptavidin, biotin, heparin, type IV collagen, protein A, and protein G.

Exemplary antibiotics include antibiotic peptides.

In still further embodiments, the biomolecule can be selected from mono-2-(carboxymethyl) hexadecanamidopoly (ethylene glycol)₂₀₀ mono-4-benzoylbenzyl ether, mono-3-carboxyheptadecanamidopoly (ethylene glycol)₂₀₀ mono-4-benzoylbenzyl ether, mono-2-(carboxymethyl) hexadecanamidotetra (ethylene glycol) mono-4-benzoylbenzyl ether, mono-3-carboxyheptadecanamidotetra (ethylene glycol) mono-4-benzoylbenzyl ether, N-[2-(4-benzoylbenzyloxy) ethyl]-2-(carboxymethyl) hexadecanamide, N-[12-(4-benzoylbenzyloxy)ethyl]-3-carboxyheptadecanamide, N-[12-(benzoylbenzyloxy) dodecyl]-2-(carboxymethyl) hexadecanamide, N-[2-(benzoylbenzyloxy) dodecyl]-3-carboxyheptadecanamide, N-[3-(4-benzoylbenzamido) propyl]-2-(carboxymethyl) hexadecanamide, N-[3-(4-benzoylbenzamido) propyl]-3-carboxyheptadecanamide, N-(3-benzoylphenyl)-2-(carboxymethyl) hexadecanamide, N-(3-benzoylphenyl)-3-carboxyheptadecanamide, N-(4-benzoylphenyl)-2-(carboxymethyl) hexadecanamide, poly(ethylene glycol)₂₀₀ mono-15-carboxypentadecyl mono-4-benzoylbenzyl ether, and mono-15-carboxypenta-decanamidopoly (ethylene glycol)₂₀₀ mono-4-benzoylbenzyl ether.

Combinations of different biocompatible agents can also be used.

In some embodiments of the invention the biomolecule is soluble in a non-aqueous solvent. For example, heparin that is soluble in a non-aqueous solvent is used, such as benzalkonium heparin. In these aspects, coating can be provided by a method that includes the steps of (a) disposing a polymeric material having a first reactive group, and (b) disposing a biomolecule having a second reactive group, wherein both the polymeric material and the biomolecule are soluble in a non-aqueous solvent, and wherein steps (a) and (b) are performed simultaneously. The biomolecule and polymeric material become coupled together via the reactive groups as they are disposed on the surface, and form a coating.

In some embodiments, one or more bioactive agents can optionally be included in the coating that includes the coupled biomolecule. A bioactive agent can be disposed on the surface of the medical device or medical item during the coating process, for example, in combination with the polymeric material and/or the biocompatible agent. The bioactive agent can be controllably released from the coating. The bioactive agent can be released from or presented by the coating once the coating is formed on the medical device and implanted in a patient. In some embodiments, the coating composition can include more than one bioactive agent, wherein each of the bioactive agents can be independently selected depending upon the desired therapeutic application of the invention.

The terms “biomolecule” and “bioactive agent” as used herein are not intended to limit to particular groups of compounds that are exclusive of one another, but rather are intended to facilitate understanding of the arrangement of features of the present coating. The term “biomolecule” refers to a molecule that is stably coupled to the polymeric material of the coating via a reactive pair, as described herein, that can provide a functional property, such as the binding of an analyte, or that can exert a biological effect, such as affecting the function of a blood cell. In many aspects of the invention the biomolecule will be a larger molecule such as a polysaccharide, polypeptide, or polynucleotide.

The term “bioactive agent” refers to a molecule that can be optionally present in the present coating and, if present, is not covalently bonded to the coating materials. In this case, the bioactive agent can be eluted or released from the coating when placed in a liquid or biological medium. For example, the bioactive agent may be released by particle dissolution or diffusion when biologically-stable matrices are used. In some cases, the bioactive agent may exert a biological effect in the same way a biomolecule that is coupled to the polymeric material of the coating exerts an effect; however, the bioactive agent is not required to remain associated with the coating. In some aspects the bioactive agent is included in the coating has a molecular weight of 1000 Da or less) and is a synthetic or naturally occurring compound, for example, the bioactive agent can be a non-polymeric compound. In other preferred aspects the bioactive agent has hydrophobic properties.

The bioactive agent can be a peptide, protein, carbohydrate, nucleic acid, lipid, polysaccharide or combinations thereof, or more preferably, a synthetic inorganic or organic molecule. The bioactive agent can cause a biological effect when administered in vivo to an animal, including but not limited to birds and mammals, including humans. Examples of bioactive agents include antigens, enzymes, hormones, receptors, peptides, and gene therapy agents. Examples of suitable gene therapy agents include a) therapeutic nucleic acids, including antisense DNA and antisense RNA, and b) nucleic acids encoding therapeutic gene products, including plasmid DNA and viral fragments, along with associated promoters and excipients. Examples of other molecules that can be incorporated include nucleosides, nucleotides, antisense, vitamins, minerals, and steroids.

In some embodiments, coatings of the present invention prepared according to this process can be used to deliver bioactive agents such as nonsteroidal anti-inflammatory compounds, anesthetics, chemotherapeutic agents, immunotoxins, immunosuppressive agents, steroids, antibiotics, antivirals, antifungals, steroidal anti-inflammatories, and anticoagulants. For example, the bioactive agent can be a biocompatible agent as described herein.

For example, hydrophobic drugs such as lidocaine or tetracaine can be included in the coating and are released over several hours.

Classes of bioactive agents which can be incorporated into biodegradable coatings (both the natural biodegradable matrix and/or the biodegradable microparticles) of this invention include, but are not limited to: ACE inhibitors, actin inhibitors, analgesics, anesthetics, anti-hypertensives, anti polymerases, antisecretory agents, anti-AIDS substances, antibiotics, anti-cancer substances, anti-cholinergics, anti-coagulants, anti-convulsants, anti-depressants, anti-emetics, antifungals, anti-glaucoma solutes, antihistamines, antihypertensive agents, anti-inflammatory agents (such as NSAIDs), anti metabolites, antimitotics, antioxidants, anti-parasite and/or anti-Parkinson substances, antiproliferatives (including antiangiogenesis agents), anti-protozoal solutes, anti-psychotic substances, anti-pyretics, antiseptics, anti-spasmodics, antiviral agents, calcium channel blockers, cell response modifiers, chelators, chemotherapeutic agents, dopamine agonists, extracellular matrix components, fibrinolytic agents, free radical scavengers, growth hormone antagonists, hypnotics, immunosuppressive agents, immunotoxins, inhibitors of surface glycoprotein receptors, microtubule inhibitors, miotics, muscle contractants, muscle relaxants, neurotoxins, neurotransmitters, opioids, photodynamic therapy agents, prostaglandins, remodeling inhibitors, statins, steroids, thrombolytic agents, tranquilizers, vasodilators, and vasospasm inhibitors.

Antibiotics are art recognized and are substances which inhibit the growth of or kill microorganisms. Examples of antibiotics include penicillin, tetracycline, chloramphenicol, minocycline, doxycycline, vancomycin, bacitracin, kanamycin, neomycin, gentamycin, erythromycin, cephalosporins, geldanamycin, and analogs thereof. Examples of cephalosporins include cephalothin, cephapirin, cefazolin, cephalexin, cephradine, cefadroxil, cefamandole, cefoxitin, cefaclor, cefuroxime, cefonicid, ceforamide, cefotaxime, moxalactam, ceflizoxime, ceftriaxone, and cefoperazone.

Antiseptics are recognized as substances that prevent or arrest the growth or action of microorganisms, generally in a nonspecific fashion, e.g., by inhibiting their activity or destroying them. Examples of antiseptics include silver sulfadiazine, chlorhexidine, glutaraldehyde, peracetic acid, sodium hypochlorite, phenols, phenolic compounds, iodophor compounds, quaternary ammonium compounds, and chlorine compounds.

Anti-viral agents are substances capable of destroying or suppressing the replication of viruses. Examples of anti-viral agents include α-methyl-P-adamantane methylamine, hydroxyethoxymethylguanine, adamantanamine, 5-iodo-2′-deoxyuridine, trifluorothymidine, interferon, and adenine arabinoside.

Enzyme inhibitors are substances that inhibit an enzymatic reaction. Examples of enzyme inhibitors include edrophonium chloride, N-methylphysostigmine, neostigmine bromide, physostigmine sulfate, tacrine HCl, tacrine, 1-hydroxymaleate, iodotubercidin, p-bromotetramisole, 10-(α-diethylaminopropionyl)-phenothiazine hydrochloride, calmidazolium chloride, hemicholinium-3,3,5-dinitrocatechol, diacylglycerol kinase inhibitor I, diacylglycerol kinase inhibitor II, 3-phenylpropargylamine, N-monomethyl-L-arginine acetate, carbidopa, 3-hydroxybenzylhydrazine HCl, hydralazine HCl, clorgyline HCl, deprenyl HCl, L(−), deprenyl HCl, D(+), hydroxylamine HCl, iproniazid phosphate, 6-MeO-tetrahydro-9H-pyrido-indole, nialamide, pargyline HCl, quinacrine HCl, semicarbazide HCl, tranylcypromine HCl, N,N-diethylaminoethyl-2,2-diphenylvalerate hydrochloride, 3-isobutyl-1-methylxanthine, papaverine HCl, indomethacin, 2-cyclooctyl-2-hydroxyethylamine hydrochloride, 2,3-dichloro-α-methylbenzylamine (DCMB), 8,9-dichloro-2,3,4,5-tetrahydro-1H-2-benzazepine hydrochloride, p-aminoglutethimide, p-aminoglutethimide tartrate, R(+), p-aminoglutethimide tartrate, S(−), 3-iodotyrosine, alpha-methyltyrosine, L(−) alpha-methyltyrosine, D L(−), cetazolamide, dichlorphenamide, 6-hydroxy-2-benzothiazolesulfonamide, and allopurinol.

Anti-pyretics are substances capable of relieving or reducing fever. Anti-inflammatory agents are substances capable of counteracting or suppressing inflammation. Examples of such agents include aspirin (salicylic acid), indomethacin, sodium indomethacin trihydrate, salicylamide, naproxen, colchicine, fenoprofen, sulindac, diflunisal, diclofenac, indoprofen and sodium salicylamide. Local anesthetics are substances that have an anesthetic effect in a localized region. Examples of such anesthetics include procaine, lidocaine, tetracaine and dibucaine.

Cell response modifiers are chemotactic factors such as platelet-derived growth factor (pDGF). Other chemotactic factors include neutrophil-activating protein, monocyte chemoattractant protein, macrophage-inflammatory protein, SIS (small inducible secreted) proteins, platelet factor, platelet basic protein, melanoma growth stimulating activity, epidermal growth factor, transforming growth factor (alpha), fibroblast growth factor, platelet-derived endothelial cell growth factor, insulin-like growth factor, nerve growth factor, and bone growth/cartilage-inducing factor (alpha and beta). Other cell response modifiers are the interleukins, interleukin inhibitors or interleukin receptors, including interleukin 1 through interleukin 10; interferons, including alpha, beta and gamma; hematopoietic factors, including erythropoietin, granulocyte colony stimulating factor, macrophage colony stimulating factor and granulocyte-macrophage colony stimulating factor; tumor necrosis factors, including alpha and beta; transforming growth factors (beta), including beta-1, beta-2, beta-3, inhibin, activin, and DNA that encodes for the production of any of these proteins.

Examples of statins include lovastatin, pravastatin, simvastatin, fluvastatin, atorvastatin, cerivastatin, rousvastatin, and superstatin.

Imaging agents are agents capable of imaging a desired site, e.g., tumor, in vivo, can also be included in the coating composition. Examples of imaging agents include substances having a label which is detectable in vivo, e.g., antibodies attached to fluorescent labels. The term antibody includes whole antibodies or fragments thereof.

Other examples of suitable bioactive agents include analogues of rapamycin (“rapalogs”), ABT-578 from Abbott, dexamethasone, betamethasone, vinblastine, vincristine, vinorelbine, poside, teniposide, dactinomycin (actinomycin D), daunorubicin, doxorubicin, idarubicin, anthracyclines, mitoxantrone, bleomycins, plicamycin (mithramycin), mitomycin, mechlorethamine, cyclophosphamide and its analogs, melphalan, chlorambucil, ethylenimines and methylmelamines, alkyl sulfonates-busulfan, nirtosoureas, carmustine (BCNU) and analogs, streptozocin, trazenes-dacarbazinine, methotrexate, fluorouracil, floxuridine, cytarabine, mercaptopurine, thioguanine, pentostatin, 2-chlorodeoxyadenosine, cisplatin, carboplatin, procarbazine, hydroxyurea, mitotane, aminoglutethimide, estrogen, heparin, synthetic heparin salts, aspirin, dipyridamole, ticlopidine, clopidogrel, breveldin, cortisol, cortisone, fludrocortisone, prednisone, prednisolone, 6U-methylprednisolone, triamcinolone, aspirin, acetaminophen, indomethacin, sulindac, etodalac, tolmetin, diclofenac, ketorolac, ibuprofen and derivatives, mefenamic acid, meclofenamic acid, piroxicam, tenoxicam, phenylbutazone, oxyphenthatrazone, nabumetone, auranofin, aurothioglucose, gold sodium thiomalate, azathioprine, mycophenolate mofetil, vascular endothelial growth factor (VEGF), fibroblast growth factor (FGF); angiotensin receptor blocker; nitric oxide donors; anti-sense oligionucleotides and combinations thereof; cell cycle inhibitors, mTOR inhibitors, and growth factor signal transduction kinase inhibitors.

A comprehensive listing of bioactive agents can be found in The Merck Index, Thirteenth Edition, Merck & Co. (2001). Bioactive agents are commercially available from Sigma Aldrich Fine Chemicals, Milwaukee, Wis.

The concentration of the bioactive agent or agents can range from about 0.01 to about 90 percent, by weight, based on the weight of the final coated composition.

The particular bioactive agent, or combination of bioactive agents, can be selected depending upon one or more of the following factors: the application of the controlled delivery device, the medical condition to be treated, the anticipated duration of treatment, characteristics of the implantation site, the number and type of bioactive agents to be utilized, and the like.

Application techniques for the coating of polymeric material include, for example, dipping, spraying, and the like. The suitability of the coating composition for use with a particular medical device, and in turn, the suitability of the application technique, can be evaluated by those skilled in the art, given the present description.

At least a portion of the surface of the article is coated with the coating composition. In some embodiments, the entire surface of the article can be coated with the coating composition. The amount of the surface area provided with the polymeric material can be determined according to such factors as the article to be utilized, and the application of the article.

In order to provide a coating the reagents described herein can be prepared in solvents, and/or dispersants individually, or in some cases, in combination.

In some aspects of the invention an adherent polymer, such as an acrylate polymer having a first reactive group, is prepared in a coating composition for application to the surface of a substrate. The composition can be used to prepare a first coated layer. Useful solvents or dispersants for these types of polymers can be selected from any of the solvents listed below.

Useful solvents or dispersants include, but are not limited to, alcohols (e.g., methanol, ethanol, n-propanol and isopropanol), alkanes (e.g., halogenated or unhalogenated alkanes such as hexane, heptane, cyclohexane, methylene chloride and chloroform), amides (e.g., dimethylformamide, N-methylpyrrolidone), ethers (e.g., tetrahydrofuran (THF), dipropyl ether and dioxolane), ketones (e.g., methyl ethyl ketone, methyl isobutyl ketone), aromatic compounds (e.g., toluene and xylene), nitriles (e.g., acetonitrile), and ester (e.g., ethyl acetate and butyl acetate).

A preferred solvent includes THF. Particularly useful ranges for an acrylate polymer having a first reactive group are at concentrations in the range of 1-100 mg/mL, more preferably in the range of 10-50 mg/mL.

After the first coated layer that includes the adherent polymer has been formed on the article surface, a composition that includes the biomolecule can be deposited on the first coated layer.

In one aspect, the biomolecule is a hydrophilic compound, such as a hydrophilic polymer. An exemplary hydrophilic polymer that can be coupled to the polymeric material of the first coated layer is heparin. The hydrophilic compound, such as heparin, can be dissolved in a suitable polar liquid, such as an aqueous phosphate or carbonate buffer, or a mixture of water and an alcohol, such as isopropanol. Useful concentrations for the biomolecule in a coating composition range from less than 1 mg/mL to about 200 mg/mL, and preferably in the range of 5 mg/mL to about 100 mg/mL mg/mL.

Therefore in some aspects a method for providing a biocompatible surface includes the steps of (a) disposing an adherent polymer on a surface, the adherent polymer comprising (i) a first reactive group; (b) disposing a biomolecule, wherein the biomolecule comprises a second reactive group that is reactive with the first reactive group. In some embodiments step (a) is performed before step (b), while in another aspect, step (a) and step (b) are performed simultaneously. The reaction between the first and second reactive group allows the biomolecule to be coupled to the surface. In preferred embodiments the adherent polymer comprises an acrylate polymer and the biomolecule comprises heparin.

In some aspects, the method can also include a step of disposing an anionic polymer, such as a sulfated polysaccharide, on the surface.

In other aspects of the invention a polymer having pendent amine groups and pendent photoreactive groups, such as photo-derivatized polylysine, polyomithine, polyethylenimine, polypropylenimine, or polyamidoamine, is prepared in a coating composition for application to the surface of a substrate. Useful solvents or dispersants for these types of polymers can be selected from:

Useful solvents or dispersants include, polar and aqueous liquids including water and water mixtures including alcohols (e.g., methanol, ethanol, n-propanol and isopropanol) and buffered aqueous solutions. Particularly useful ranges for the polymer having pendent amine and photoreactive groups are at concentrations in the range of 1-100 mg/mL.

In one aspect, the method for preparing a biocompatible surface comprises the steps of (a) disposing a polymer on a surface, the polymer selected from the group consisting of polylysine, polyornithine, polyethylenimine, polypropylenimine, and polyamidoamine, and comprising (i) a photoreactive group selected from photoreactive acetophenone, benzophenone, anthraquinone, anthrone, and anthrone-like heterocycles; (b) disposing a biomolecule, wherein the biomolecule comprises an amine-reactive group; and (c) treating the polymer to activate the photoreactive group to bind the polymeric material to the surface. In this aspect, the polymer selected from the group consisting of polylysine, polyornithine, polyethylenimine, polypropylenimine, and polyamidoamine has an amine group that reacts with the amine-reactive group on the biomolecule, thereby coupling the biomolecule to the surface.

In another aspect, the method for preparing a biocompatible surface comprises the steps of (a) disposing a polymer on a surface, the polymer selected from the group consisting of polylysine, polyomithine, polyethylenimine, polypropylenimine, and polyamidoamine, and comprising (i) a photoreactive group selected from photoreactive acetophenone, benzophenone, anthraquinone, anthrone, and anthrone-like heterocycles; (b) disposing an anionic polymer; (c) disposing biomolecule, wherein the biomolecule comprises an amine-reactive group; and (d) treating the polymer to activate the photoreactive group to bind the polymeric material to the surface. Preferably, the anionic polymer is dextran sulfate.

In preferred embodiments the photo-derivatized polylysine, polyornithine, polyethylenimine, polypropylenimine, and polyamidoamine couples an heparin via an aldehyde group.

The invention also includes use of medical devices having a biocompatible coating as described herein. It will be apparent from the specification that devices having coatings as described can be used in a wide variety of procedures or processes, and that the invention also encompasses the use of these devices in these procedures.

The invention will be further described with reference to the following non-limiting Examples.

Heparin Activity Assay

The antithrombotic activity of heparin is due to its inhibition of thrombin, which is a protease that is known to participate in the clotting cascade. Heparin inhibits thrombin activity by first binding to antithrombin III (ATIII). The heparin/ATIII complex then binds to and inactivates thrombin, after which the heparin is released and can bind to another ATIII. The assay for inhibition of thrombin by immobilized heparin was conducted by measuring the cleavage of a chromogenic peptide substrate by thrombin.

Prior to performing the Heparin Activity Assay, substrates (such as polypropylene plates or stents) were washed overnight (12-18 hours) to remove any unbound material from the substrates. Substrates were washed in diH₂O or PBS at a temperature of about 37° C. on an orbital shaker (set for gentle agitation).

Each assay was conducted in 1 mL of PBS that contained 0.85 mg BSA (Sigma Chemical Co.), 10 mU human thrombin (Sigma Chemical Co.), 100 mU/mL ATIII (Baxter Biotech, Chicago, Ill.), and 0.17 μmole of the chromogenic thrombin substrate S-2238 (Kabi Pharmacia, Franklin, Ohio). To this assay solution was added either uncoated or heparin coated substrates (to evaluate heparin activity) or standard concentrations of heparin (to generate standard curves of heparin content versus absorbance). For standard curves, the amounts of heparin that were added ranged from 2.5 mU to 25 mU. The color generated, measured as absorbance at 405 nm, by thrombin mediated cleavage of the S-2238 was read using a spectrophotometer after 2 hours of incubation at 37° C. The absorbance was directly related to the activity of the thrombin and, thus, inversely related to the amount of activation of ATIII induced by the heparin in solution or immobilized on the surface of the substrate. Activity of surface bound heparin was calculated by comparing the absorbance values generated to the absorbance values generated with known amounts of added heparin.

Commercial preparations of heparin are commonly calibrated in USP units, 1 unit being defined as the quantity that prevents 1.0 mL of citrated sheep plasma from clotting for 1 h after the addition of 0.2 mL of 10 g/L CaCl₂ (see Majerus P W, et al. Anticoagulant, thrombolylic, and antiplatelet drugs. In: Hardman J G, Limbrid L E, eds., Goodman and Gilman's The pharmacological bases of therapeutics, 9th ed, New York: McGraw Hill, 1996:1341-6). Commercial preparations of heparin typically include the heparin activity of the preparation. In order to determine the heparin activity of a heparin coating described herein, the above assay can be performed and compared to a standard generated from a commercial preparation of heparin, based on the above definition of heparin activity.

### EXAMPLE 1

### Preparation of N-(3-isothiocyanatopropyl)-2-methylacrylamide (APMA-NCS)

An isothiocyanate (NCS) methacrylamide monomer was prepared in the following manner. Solution (A) was made by placing APMA (N-{3-Aminopropyl}methacrylamide hydrochloride; the preparation of which is described in U.S. Pat. No. 6,465,178) 1.00 g (5.60 mmole), chloroform (5.0 ml), and carbon disulfide 2.0 ml (6.46 mmole) in a vial. Solution B was made by placing dicyclohexylcarbodiimide (“DCC”), 1.29 g (6.25 mmole) in a vial and dissolving in 2.0 ml of chloroform. Solutions A and B were placed in an ice bath and then solution (B) was added to solution (A). The mixture was then shaken for 2 hours at room temperature. The product was isolated (flash purified) using a silica column 25 mm in diameter and 190 mm long. The column was eluted with 50-12 ml fractions of a chloroform/acetone mixture at a ratio of 96:4. Fractions 12 to 21 were combined and evaporated to give about 800 mg oil (having some solid). The oil was dissolved in 2 ml acetone and 2 ml chloroform and pipet filtered to remove the precipitate. TLC (thin layer chromatography) was performed using either 5% acetone in chloroform or 10% methanol in chloroform and indicated that the reaction product was a single compound. GLC (gas liquid chromatography) analysis indicated the product to be >90% pure. NMR analysis at 400 MHz was consistent with the desired product: ¹H NMR (CDCl₃) amide proton 6.11 (b, 1H), vinyl protons 5.72, 5.37 (d, 2H), methylene protons adjacent to amide N 3.62 (m, 2H), methylene protons adjacent to NCS 3.45 (m, 2H), and central methylene protons along with the methyl protons 2.00 (m, 5H).

### EXAMPLE 2

### Preparation of Butyl Methacrylate/APMA-NCS Copolymers (pBMA-NCS)

Copolymers having butyl methacrylate monomeric units and pendent isothiocyanate groups (pBMA-NCS) were prepared by copolymerizing BMA monomers with APMA-NCS monomers (as synthesized in Example 1) at varying molar ratios.

To provide pBMA-(5%)NCS the following procedure was performed. Kollidon™ K-90 (BASF), 20 mg (0.1 pph), was added to 100 mL of water and heated to 65° C. with vigorous stirring and deoxygenated with a nitrogen gas sparge. 2,2′-azobis(2,4-dimethylpentanenitrile) (Vazo™ 52; DuPont) 380 mg (1.53 mmoles), and APMA-NCS, 1.28 g (6.95 mmoles; as prepared in Example 1) were dissolved in 20.9 mL (95 mole %; 0.13 moles) of butyl methacrylate with stirring. Once the water/Kollidon solution stabilized at 65° C., the Vazo™ 52/APMA-NCS/butyl methacrylate solution was added with vigorous stirring. The reaction proceeded for 45 minutes and was then quenched with deionized water. The reaction solution was filtered through a mesh screen and the product beads were washed with 200 mL of methanol for 3 hrs. The beads were isolated by filtration and dried under vacuum to give 16.3 g of product. IR analysis confirmed the presence of the NCS group at 2186 and 2112 cm⁻¹.

To provide pBMA-(10%)NCS, APMA-NCS, 2.52 g (13.68 mmoles; as prepared in Example 1), and 19.56 mL of butyl methacrylate (0.12 moles) were substituted for the amounts of APMA-NCS and BMA described above.

To provide pBMA-(20%)NCS, APMA-NCS, 4.89 g (26.54 mmoles; as prepared in Example 1), 16.90 mL of butyl methacrylate (0.11 moles) and 370 mg of Vazo™ 52 (1.49 mmoles) were substituted for the amounts of APMA-NCS, BMA and Vazo 52 described above.

### EXAMPLE 3

### Preparation of a Butyl Methacrylate/glycidyl Methacrylate Copolymer (pBMA-Epoxide)

Copolymers having butyl methacrylate monomeric units and pendent oxirane (epoxide) groups (pBMA-epoxide) were prepared by copolymerizing BMA monomers with glycidyl methacrylate monomers at varying molar ratios.

To provide pBMA-(10%)epoxide the following procedure was performed. Butyl methacrylate, 50.34 mls (0.32 moles), was dissolved in 168.73 mL of tetrahydrofuran (THF), followed by 4.80 mL (0.035 moles) of glycidyl methacrylate with stirring. This reaction solution was deoxygenated with nitrogen and heated to 60° C. Once the reaction solution had stabilized at 60° C., 0.022 mL (0.0003 moles) mercaptoethanol, and 970 mg (0.0039 moles) of Vazo™ 52 was added. The reaction was allowed to proceed with stirring under nitrogen at 60° C. for four hours. After this time, half of the reaction solution was slowly dripped into 1.5 liters of methanol (MeOH) and stirred very vigorously and the other half into 1.5 liters of hexanes and stirred vigorously. The precipitated product was isolated using a mesh screen and dried under vacuum to give 12.32 g from MeOH ppt and 11.67 g from hexanes. NMR analysis confirmed the presence of the epoxy group at 2.65, 2.85, and 3.2 ppm. The epoxide portion of the polymer was roughly 12.5 mole % (theoretical, 10 mole %).

To provide pBMA-(5%)epoxide, glycidyl methacrylate, 2.50 g (17.58 mmoles), and 53.13 mL of butyl methacrylate (0.33 moles) were substituted for the amounts of glycidyl methacrylate and BMA described above.

To provide pBMA-(20%)epoxide, glycidyl methacrylate, 10.00 g (70.32 mmoles), and 44.74 mL of butyl methacrylate (0.28 moles) were substituted for the amounts of glycidyl methacrylate and BMA described above.

### EXAMPLE 4

### Preparation of a Butyl Methacrylate/MAL-EAC-NOS Copolymer (pBMA-NOS)

Copolymers having butyl methacrylate monomeric units and pendent N-oxysuccinimide (NOS) groups (pBMA-NOS) were prepared by copolymerizing BMA monomers with NOS-containing monomers at varying molar ratios.

To provide pBMA-(10%)NOS the following procedure was performed.

Butyl methacrylate, 50.34 mL (0.32 moles), was dissolved in 168.73 mL of tetrahydrofuran (THF), followed by 9.71 g (0.031 moles) of MAL-EAC-NOS(N-succinimidyl 6-maleimidohexanoate, the synthesis of which is described in Example 4 of U.S. Pat. No. 5,858,653 (Duran et al.)) with stirring. This reaction solution was deoxygenated with nitrogen and heated to 60° C. Once the reaction solution had stabilized at 60° C., 0.022 mL (0.0003 moles) mercaptoethanol, and 870 mg (0.0035 moles) of Vazo™ 52 was added. The reaction was allowed to proceed with stirring under nitrogen at 60° C. for four hours. After this time, half of the reaction solution was slowly dripped into 1.5 liters of methanol (MeOH) stirred very vigorously and the other half into 1.5 liters of hexanes stirred vigorously. The precipitated product was isolated using a mesh screen and dried under vacuum to give 6.58 g from MeOH ppt and 11.22 g from hexanes.

To provide pBMA-(5%)NOS, MAL-EAC-NOS, 5.12 g (16.61 mmoles), 50.20 mL of butyl methacrylate (0.32 moles), and 920 mg of Vazo™ 52 (3.70 mmoles) were substituted for the amounts of MAL-EAC-NOS, BMA and Vazo™ 52 described above. The material was precipitated using just MeOH.

To provide pBMA-(20%)NOS, MAL-EAC-NOS, 17.57 g (56.99 mmoles), 36.27 mL of butyl methacrylate (0.23 moles), and 790 mg of Vazo™ 52 (3.18 mmoles) were substituted for the amounts of MAL-EAC-NOS, BMA, and Vazo™ 52 described above. The material was precipitated using just MeOH.

### EXAMPLE 5

### Preparation of a Butyl Methacrylate/Trimethylsilane Copolymer (pBMA-TMS)

Copolymers having butyl methacrylate monomeric units and pendent trimethylsilane (TMS) groups (pBMA-TMS) were prepared by copolymerizing BMA monomers with TMS-containing monomers (a TMS ester of methacrylic acid) at varying molar ratios.

To provide pBMA-(10%)TMS the following procedure was performed.

Butyl methacrylate, 49.77 mL (0.31 moles), was dissolved in 168.73 mL of tetrahydrofuran (THF), followed by 5.50 g (0.035 moles) of trimethylsilyl methacrylate with stirring. This reaction solution was deoxygenated with nitrogen and heated to 60° C. Once the reaction solution had stabilized at 60° C., 0.022 mL (0.0003 moles) mercaptoethanol, and 960 mg (0.0039 moles) of Vazo™ 52 was added. The reaction was allowed to proceed with stirring under nitrogen at 60° C. for three hours. After this time, half of the reaction solution was slowly dripped into 1.5 liters of methanol (MeOH) stirred very vigorously and the other half was triturated with MeOH. The precipitated product was isolated using a mesh screen and dried under vacuum to give 20.87 g from MeOH ppt and 4.88 g from trituration.

To provide pBMA-(5%)TMS the following procedure was performed. Kollidon™ K-90 (BASF), 20 mg (0.1 pph), was added to 110 mL of water and heated to 65° C. with vigorous stirring and deoxygenated with a nitrogen gas sparge. 2,2′-azobis(2,4-dimethylpentanenitrile) (Vazo™ 52) 390 mg (1.57 mmoles), and trimethylsilyl methacrylate, 1.11 g (7.01 mmoles) were dissolved in 21.13 mL (95 mole %; 0.133 moles) of butyl methacrylate with stirring. Once the water/Kollidon solution stabilized at 65° C., the Vazo™ 52/TMS-methacrylate/butyl methacrylate solution was added with vigorous stirring. The reaction proceeded for 45 minutes and was then quenched with deionized water. The reaction solution was filtered through a mesh screen and the product beads were washed with 200 mL of methanol. The beads were isolated by filtration and dried under vacuum to give 15.6 g of product.

### EXAMPLE 6

### Preparation of PEI-BBA

A photoderivatived polymer having pendent amine groups was prepared.

Polyethylenimine (PEI; 24.2 wt. % solids; 2000 kg/mol Mw; BASF Corp.) was dried under vacuum and 1.09 g PEI was dissolved in a 19 mL of 90:10 (v/v) chloroform:methanol solution. The PEI solution was then chilled to 0° C. in an ice bath. In 2.8 mL chloroform was added 62 mg BBA-Cl (4-benzoylbenzoyl chloride; the preparation of which is described in U.S. Pat. No. 5,858,653) which was allowed to dissolve. Add the BBA-Cl solution to the chilled, stirring PEI solution. Allow the reaction solution to stir overnight while warming to room temperature (TLC analysis of the reaction solution revealed no unreacted BBA-Cl present after 2.5 hrs.). The next day the reaction solution was transferred into a large flask and one equivalent of concentrated hydrochloric acid was added along with 77.5 mL deionized water. The organic solvents were removed under vacuum at 40° C. until the aqueous PEI solution was clear in appearance. The aqueous PEI solution was then diluted to a final concentration of 10 mg/mL for use as a coating solution.

### EXAMPLE 7

### Preparation of PEI-BBA-Heparin Coated Substrates

A solution of sodium heparin (Solution A) was prepared by dissolving 10 g of lyophilized sodium heparin (179 U/mg activity; Celsus Laboratories, Inc.) in 150 mL deionized water. The pH of the aqueous sodium heparin solution was adjusted to 7.0. The solution was then stored at 4° C. Next, sodium periodate (0.401 g, 1.87 mmol; Sigma-Aldrich, Inc.) was dissolved in Solution A and stirred at for one hour at 4° C. A solution of PEI-BBA (Solution B) was also prepared by diluting 100 mL of 10 mg/mL PEI-BBA (Example 6) in water with 50 mL deionized water and adjusting the pH to 9.0. Potassium phosphate di-basic (5.2 g, 29.9 mmol; Sigma-Aldrich, Inc.) was added to Solution B and the pH was adjusted to pH 9.0. Solution A was then poured into Solution B and sodium cyanoborohydride (3.756 g, 59.8 mmol; Sigma-Aldrich, Inc.) was added to the combined solution. The combined solution was stirred overnight at room temperature followed by dialysis in 50,000 MWCO dialysis tubing against PBS solution (one day) and then deionized water (two days). The dialyzed solution was then lyophilized to yield 6.471 g of PEI-BBA-heparin material.

A 25 mg/mL coating solution of PEI-BBA-heparin in (60:40) v/v deionized water:isopropanol was prepared and used to dip-coat a Pebax rod after a receiving a basecoat of photo-derivatized poly(vinylpyrrolidone) (photo-PVP) as prepared as described in U.S. Pat. No. 5,637,460. The Pebax rod was then cut into three pieces each having an approximate surface area of 1 cm². The three coated rods were assayed for heparin activity against three uncoated Pebax rods (each approximately 1 cm² surface area) with results shown in the Table 2 below.

TABLE 2

| | Abs | Activity | Mean | Std. |
|---|---|---|---|---|
| Sample | (A.U.) | (mU/cm²) | (mU/cm²) | Dev. |
| Coated Pebax 1 | 0.345 | 13 | 11 | 1 |
| Coated Pebax 2 | 0.374 | 11 | N/A | N/A |
| Coated Pebax 3 | 0.370 | 11 | N/A | N/A |
| Uncoated Pebax 1 | 0.472 | 3 | 3 | 0 |
| Uncoated Pebax 2 | 0.472 | 3 | N/A | N/A |
| Uncoated Pebax 3 | 0.473 | 3 | N/A | N/A |

### EXAMPLE 8

### Preparation of N-{3-[(bromoacetyl)amino]propyl}-2-methylacrylamide (Bromoacetyl-APMA)

APMA (N-{3-Aminopropyl}methacrylamide hydrochloride; the preparation of which is described in Example 2 of U.S. Pat. No. 6,465,178) 34.0 g (0.190 mole) and chloroform (340 ml) was placed in a 1 liter flask fitted with an overhead stirrer, addition funnel, drying tube, and thermometer. The reaction mixture was cooled using a dry ice bath to −50° C. Bromoacetyl bromide, 40.4 g (0.200 mole) was added to the reaction flask over 35 minutes while the flask was kept at a temperature below −50° C. (The bromoacetyl bromide is available as a mixture of bromo- and chloro-compounds, but will be referred to herein as bromoacetyl bromide.) Finally, triethyl amine, 57 ml (41.5 g; 0.41 mole) was added over one hour at a temperature of −40° C. to −60° C. The reaction was allowed to come to room temperature (RT), phenothiazine (50 mg) was added, and the reaction was stirred overnight at RT. The reaction was placed in a separatory funnel with water (1700 ml), ice (500 g), and HCl (34 ml; 12M). The organic layer was separated and the aqueous layer was extracted with 2×300 ml chloroform. The combined organic layers were dried over sodium sulfate. The solvent was removed on a rotary evaporator at 40° C. using a water aspirator with an air bleed to give 18 g of a dark viscous liquid, which was used to prepare a haloacetyl functional polymer in Example 9. A sample of the crude dark oil was flash purified on a silica gel column, that was eluted with acetone/chloroform-30/70. Analysis on an NMR spectrometer was consistent with a mixture of bromoacetyl-APMA and chloroacetyl-APMA: ¹H NMR (CDCl₃) amide protons 7.34, 6.73 (b, 2H), vinyl protons 5.77, 5.35 (m, 2H), chloroacetyl protons 4.06 (s, fraction of 2H), bromoacetyl protons 3.87 (s, fraction of 2H), methylene protons adjacent to amides N's 3.35 (m, 4H), methyl protons 1.98 (s, sH), and central methylene protons 1.71 (m, 2H).

### EXAMPLE 9

### Preparation of a Butyl Methacrylate/Bromoacetyl Copolymer (pBMA-BA)

Copolymers having butyl methacrylate monomeric units and pendent bromoacetyl (BA) groups (pBMA-BA) were prepared by copolymerizing BMA monomers with BA-containing monomers at varying molar ratios.

To provide pBMA-(10%) BA the following procedure was performed. Butyl methacrylate, 27.83 mL (0.175 moles), was dissolved in 101.24 mL of tetrahydrofuran (THF), followed by 5.12 g (0.0195 moles) of bromoacetyl-APMA with stirring. This reaction solution was deoxygenated with nitrogen and heated to 60° C. Once the reaction solution had stabilized at 60° C., 0.022 mL (0.0003 moles) mercaptoethanol, and 540 mg (0.00217 moles) of Vazo™ 52 was added. The reaction was allowed to proceed with stirring under nitrogen at 60° C. for two hours. After this time, half of the reaction solution was slowly dripped into 1.5 liters of methanol (MeOH) stirred very vigorously and the other half was dripped into hexanes. The precipitated product was isolated using a mesh screen and dried under vacuum to give 2.37 g from MeOH ppt and 4.67 g from hexanes ppt.

### EXAMPLE 10

### Preparation of a Butyl Methacrylate/Aminopropylmethacrylamide Copolymer (PBMA-APMA)

Copolymers having butyl methacrylate and N-(3-aminopropyl) methacrylamide monomeric units (pBMA-APMA) were prepared according to the following procedure:

The N-(3-aminopropyl) methacrylamide hydrochloride (APMA-HCl) was free based by dissolving 25 g (0.140 moles) in a solution of 70 g of potassium carbonate (K₂CO₃) in 500 mls deionized water. The aqueous solution was extracted with 200 mls chloroform (CHCl₃) three times. The organic phases were pooled and dried over sodium sulfate (Na₂SO₄). The solvent was removed in vacuo.

To provide pBMA-(10%) NH₂ the following procedures were performed. Butyl methacrylate, 50.34 mL (0.32 moles), was dissolved in 168.73 mL of tetrahydrofuran (THF), followed by 5.00 g (35.19 mmoles) of APMA (prepared above) with stirring. This reaction solution was deoxygenated with nitrogen and heated to 60° C. Once the reaction solution had stabilized at 60° C., 970 mg (3.90 mmoles) of Vazo™ 52 was added. The reaction was allowed to proceed with stirring under nitrogen at 60° C. for five hours. After this time, the reaction solution was slowly dripped into 1.5 liters of methanol (MeOH) and stirred very vigorously. The precipitated product was isolated using a mesh screen and dried under vacuum to give 19.65 g from MeOH ppt.

To provide pBMA-(20%) APMA, APMA, 9.99 g (70.3 mmoles), and 44.75 mL of butyl methacrylate (0.28 moles) were substituted for the amounts of APMA, and BMA described above.

Kollidon™ K-90 (BASF), 30 mg (0.1 pph), was added to 150 mL of water and heated to 65° C. with vigorous stirring and deoxygenated with a nitrogen gas sparge. 2,2′-azobis(2,4-dimethylpentanenitrile) (Vazo™ 52) 580 mg (2.33 mmoles), and APMA (prepared above), 3.00 g (21.11 mmoles) were dissolved in 30.20 mL (90 mole %; 0.190 moles) of butyl methacrylate with stirring. Once the water/Kollidon solution stabilized at 65° C., the Vazo™ 52/APMA/butyl methacrylate solution was added with vigorous stirring. The reaction proceeded for 60 minutes and was then quenched with deionized water. The reaction solution was washed with 200 mL of methanol. The material was isolated by decanting the MeOH and dried under vacuum to give 20.93 g.

To provide pBMA-(20%) APMA, APMA, 6.00 g (42.22 mmoles), and 26.85 mL of butyl methacrylate (0.17 moles) were substituted for the amounts of APMA, and BMA described above.

### EXAMPLE 11

### Preparation of a butyl methacrylate/N-[N′-(t-butyloxycarbonyl)-3-aminopropyl]-methacrylamide copolymer (pBMA-(APMA-tBOC))

Copolymers having butyl methacrylate and N-[N′-(t-butyloxycarbonyl)-3-aminopropyl]-methacrylamide or tert-butyl[3-(methacryloylamino)propyl]carbamate monomeric units (pBMA-(APMA-tBOC)) were prepared according to the following procedure:

To provide pBMA-(10%) APMA-t-BOC the following procedures were performed. Butyl methacrylate, 46.94 mL (0.30 moles), was dissolved in 168.73 mL of tetrahydrofuran (THF), followed by 8.03 g (32.78 mmoles) of APMA-t-BOC(N-[N′-(t-butyloxycarbonyl)-3-aminopropyl]-methacrylamide); the preparation of which is described as an intermediate in Example 2 of U.S. Pat. No. 6,465,178, with stirring.

This reaction solution was deoxygenated with nitrogen and heated to 60° C. Once the reaction solution had stabilized at 60° C., 910 mg (3.66 mmoles) of Vazo™ 52 was added. The reaction was allowed to proceed with stirring under nitrogen at 60° C. for five hours. After this time, the reaction solution was slowly dripped into 1.5 liters of methanol (MeOH) and stirred very vigorously. The precipitated product was isolated using a mesh screen and dried under vacuum.

To provide pBMA-(20%) APMA-t-BOC, APMA-t-BOC, 15.05 g (61.43 mmoles), and 39.09 mL of butyl methacrylate (0.25 moles) were substituted for the amounts of APMA, and BMA described above.

Kollidon™ K-90 (BASF), 30 mg (0.1 pph), was added to 150 mL of water and heated to 65° C. with vigorous stirring and deoxygenated with a nitrogen gas sparge. 2,2′-azobis(2,4-dimethylpentanenitrile) (Vazo™ 52) 540 mg (2.17 mmoles), and APMA-t-BOC, 4.82 g (19.67 mmoles) were dissolved in 25 mls CHCl₃ and 28.17 mL (90 mole %; 0.18 moles) of butyl methacrylate with stirring. Once the water/Kollidon solution stabilized at 65° C., the Vazo™ 52/APMA-t-BOC/butyl methacrylate solution was added with vigorous stirring. The reaction proceeded for 60 minutes and was then quenched with deionized water. The reaction solution was washed with 200 mL of methanol. The material was isolated by decanting the MeOH and dried under vacuum.

To provide pBMA-(20%) APMA-t-BOC, APMA-t-BOC, 9.03 g (36.86 mmoles), and 23.45 mL of butyl methacrylate (0.15 moles) were substituted for the amounts of APMA-t-BOC, and BMA described above.

### EXAMPLE 12

### Preparation of Blended PBMA-pCDI Coating Solution

Polycarbodiimide (pCDI; 50 wt. % solids in propylene glycol methyl ether acetate; Sigma-Aldrich, Inc.) was dried under vacuum to remove the propylene glycol methyl ether acetate solvent and 0.824 g of pCDI (neat) was dissolved in 19.8 mL tetrahydrofuran. Then, 0.817 g of poly(butyl methacrylate) (300 kg/mol M_{W}) was massed into a 20 mL amber vial and the pCDI/THF solution was poured into the vial to dissolve the pBMA. The vial was placed on a shaker to mix for 20 minutes to fully go into solution.

### EXAMPLE 13

### Preparation of a Heparin Coated Stent (Two-Step Method)

The (50:50) w/w pBMA:pCDI coating solution (Example 12) was applied to Parylene-coated stents using a spray coating procedure. The spray-coated stents were then allowed to air dry overnight. After drying, the stents were incubated in a 100 mg/mL sodium heparin solution in acetate buffer (141 mM, pH 5.5) overnight followed by a PBS wash to remove unbound heparin. The stents were then assayed for heparin activity with results shown in Table 3.

TABLE 3

| Sample | Activity (mU/cm²) | Mean (mU/cm²) | Std. Dev. |
|---|---|---|---|
| Coated Stent 1 | 19.2 | 17.4 | 2.5 |
| Coated Stent 2 | 15.6 | N/A | N/A |
| Uncoated Stent 1 | 2.9 | 3.3 | 0.6 |
| Uncoated Stent 2 | 3.7 | N/A | N/A |

### EXAMPLE 14

### Preparation of a Heparin Coated Stent (One-Step Method)

A solution of 32 mg/mL heparin-benzalkonium (Sigma-Aldrich, Inc.) is prepared in (80:20) v/v THF:IPA. An equal volume of (50:50) w/w pBMA:pCDI coating solution (as prepared in Example 12) is co-sprayed with the heparin-benzalkonium from separate reservoirs using two different spray heads simultaneously onto Parylene-caoted stents. The stent are allowed to dry overnight with solvent evaporation.

### EXAMPLE 15

### Preparation of pBMA-NCS-Heparin Coatings

Coating solutions were prepared individually containing pBMA-(5%)NCS, pBMA-(10%)NCS, and pBMA-(20%)NCS as prepared in Example 2. The pBMA-NCS copolymers were dissolved in tetrahydrofuran (THF) at 20 mg/mL.

96-well polypropylene plates were coated using 25 μL of each pBMA-NCS solution per well. The solutions were allowed to dry by evaporation of THF at room temperature.

Sodium heparin (Celsus Laboratories, Cincinnati, Ohio) was then dissolved in a 50 mM sodium phosphate (pH 8.5) solution at 10 mg/mL. 50 μL of the heparin solution was added to each well and allowed to incubate at room temperature for greater than sixteen hours. After this time, the plates were rinsed extensively with deionized water and allowed to dry. The plates were tested using a toluidine blue assay and a heparin assay. The results of heparin activity are summarized in Table 4 below.

TABLE 4

| | Heparin Activity (mU/cm²) | | | | Heparin Activity |
|---|---|---|---|---|---|
| First Coat | (a) | (b) | (c) | (d) | (mU/cm²) Ave. |
| pBMA-(5%)NCS | 10 | 5 | 4 | 3 | 5.5 |
| pBMA-(10%)NCS | 5 | | | | 5 |
| pBMA-(20%)NCS | 5 | | | | 5 |

### EXAMPLE 16

### Preparation of pBMA-Epoxide-Heparin Coatings

Coating solutions were prepared individually containing pBMA-(5%)epoxide, pBMA-(10%)epoxide, and pBMA-(20%)epoxide as prepared in Example 3. The pBMA-epoxide copolymers were dissolved in tetrahydrofuran (THF) at 20 mg/mL.

96-well polypropylene plates were coated using 25 μL of the pBMA-epoxide polymer solutions per well and allowed to dry. The sodium heparin was then dissolved in a 50 mM sodium phosphate (pH 8.5) solution at 10 mg/mL. 50 μL of the heparin solution was added to each well and allowed to incubate at room temperature for greater than sixteen hours. After this time, the plates were rinsed extensively with deionized water and allowed to dry. The plates were tested using a toluidine blue assay and a heparin assay.

Heparin activities were tested all coating solutions and the results are shown in Table 5 below.

TABLE 5

| | Heparin Activity (mU/cm²) | | | | Heparin Activity |
|---|---|---|---|---|---|
| First Coat | (a) | (b) | (c) | (d) | (mU/cm²) Ave. |
| pBMA-(5%)epoxide | 5 | 2 | | | 3.5 |
| pBMA-(10%)epoxide | 15 | 6 | 5 | 2 | 7 |
| pBMA-(20%)epoxide | 6 | 2 | 5 | | 4.3 |

### EXAMPLE 17

### Preparation of pBMA-NOS-Heparin Coatings

Coating solutions were prepared individually containing pBMA-(5%)NOS, pBMA-(10%) NOS, and pBMA-(20%)NOS as prepared in Example 4. The pBMA-NOS copolymers were dissolved in tetrahydrofuran (THF) at 20 mg/mL.

96-well polypropylene plates were coated using 25 μl of the pBMA-NOS polymer solutions per well and allowed to dry. The sodium heparin was then dissolved in a 50 mM sodium phosphate (pH 8.5) solution at 10 mg/ml. 50 μl of the heparin solution was added to each well and allowed to incubate at room temperature for greater than sixteen hours. After this time, the plates were rinsed extensively with deionized water and allowed to dry. The plates were tested using a toluidine blue assay and a heparin assay. Heparin activities were tested all coating solutions and the results are shown in Table 6 below.

TABLE 6

| | Heparin Activity (mU/cm²) | | | | Heparin Activity |
|---|---|---|---|---|---|
| First Coat | (a) | (b) | (c) | (d) | (mU/cm²) Ave. |
| pBMA-(5%)NOS | 5 | 3 | | | 4 |
| pBMA-(10%)NOS | 18 | 8 | 5 | 3 | 8.5 |
| pBMA-(20%)NOS | 7 | 3 | 3 | | 4.3 |

### EXAMPLE 18

### Preparation of pBMA-TMS-Heparin Coatings

A coating solution containing pBMA-(10%)TMS (as prepared in Example 5) was dissolved in tetrahydrofuran (THF) at 20 mg/mL.

96-well polypropylene plates were coated using 25 μl of the pBMA-TMS polymer solutions per well and allowed to dry. The sodium heparin was then dissolved in a 50 mM sodium phosphate (pH 8.5) solution at 10 mg/ml. 50 μl of the heparin solution was added to each well and allowed to incubate at room temperature for greater than sixteen hours. After this time, the plates were rinsed extensively with deionized water and allowed to dry. The plates were tested using a toluidine blue assay and a heparin assay. A heparin activity of 6 mU/cm² was observed.

### EXAMPLE 19

### Preparation of pBMA-NCS-Heparin Coatings

A coating solution containing pBMA-(20%)NCS (as prepared in Example 2) was dissolved in tetrahydrofuran (THF) at 30 mg/ml. 12-well polypropylene plates were coated using 2.5 mL of the pBMA-(20%)NCS polymer solution per well and allowed to dry. The sodium heparin was then dissolved in a 100 mM carbonate buffer solution (pH 9.0) at 20 mg/mL. 3 mL of the heparin solution was added to each well and allowed to incubate at 55° C. with agitation for sixteen hours. After this time, the plates were rinsed with deionized water, soaked in deionized water for sixteen hours and allowed to dry. The plates were tested using a heparin assay. Heparin activities of 7, 5, and 5 mU (repeated in triplicate) were observed.

### EXAMPLE 20

### Preparation of pBMA-Epoxide-Heparin Coatings

A coating solution containing pBMA-(20%)epoxide (as prepared in Example 3) was dissolved in tetrahydrofuran (THF) at 30 mg/mL. 12-well polypropylene plates were coated using 2.5 mL of the pBMA-epoxide polymer solution per well and allowed to dry. The sodium heparin was then dissolved in a 100 mM carbonate buffer solution (pH 9.0) at 20 mg/mL. 3 mL of the heparin solution was added to each well and allowed to incubate 55° C. with agitation for sixteen hours. After this time, the plates were rinsed with deionized water, soaked in deionized water for sixteen hours and allowed to dry. The plates were tested using a heparin assay. Heparin activities of 8, 8, and 8 mU (repeated in triplicate) were observed.

### EXAMPLE 21

### Preparation of pBMA-NOS-Heparin Coatings

A coating solution containing pBMA-(20%)NOS (as prepared in Example 4) was dissolved in tetrahydrofuran (THF) at 30 mg/mL. 12-well polypropylene plates were coated using 2.5 mL of the pBMA-NOS polymer solutions per well and allowed to dry. The sodium heparin was then dissolved in a 100 mM carbonate buffer solution (pH 9.0) at 20 mg/mL. 3 mL of the heparin solution was added to each well and allowed to incubate 55° C. with agitation for sixteen hours. After this time, the plates were rinsed with deionized water, soaked in deionized water for sixteen hours and allowed to dry. The plates were tested using a heparin assay. Heparin activities of 33, 15, and 13 mU (repeated in triplicate) were observed.

### EXAMPLE 22

### Preparation of pBMA-TMS-Heparin Coatings

A coating solution containing pBMA-(10%)TMS (as prepared in Example 5) was dissolved in tetrahydrofuran (THF) at 30 mg/mL. 12-well polypropylene plates were coated using 2.5 mL of the pBMA-TMS polymer solutions per well and allowed to dry. The sodium heparin was then dissolved in a 100 mM carbonate buffer solution (pH 9.0) at 20 mg/mL. 3 mL of the heparin solution was added to each well and allowed to incubate 55° C. with agitation for sixteen hours. After this time, the plates were rinsed with deionized water, soaked in deionized water for sixteen hours and allowed to dry. The plates were tested using a heparin assay. Heparin activities of 5, 5, and 6 mU (repeated in triplicate) were observed.

### EXAMPLE 23

### Preparation of pBMA-NOS-Antibody Coating

A coating solution was prepared containing pBMA-(20%) NOS as prepared in Example 4. The pBMA-NOS copolymer was prepared at 10 mg/mL in THF. Parylene-coated stainless steel disks were dip coated in copolymer solution and air dried in a fume hood. The disks were transferred to an antibody solution (0.08 mg/mL mouse IgG in 0.1 M sodium phosphate pH 8.0). The disks were incubated for 20 hours then removed and washed 5× with PBS.

The presence of antibody on the disks was elucidated by a FITC-labeled goat anti-mouse IgG.

1. An article having a biomolecule-containing coating, the coating comprising:
(a) a coated layer comprising an acrylate polymer comprising a pendent first reactive group; and
(b) a coated layer comprising a biomolecule, wherein the biomolecule is coupled to the acrylate polymer via the first reactive group.

2. The article of claim 1 wherein the acrylate polymer is a selected from the group of alkyl(meth)acrylate polymers and aromatic(meth)acrylate polymers.

3. The article of claim 2 wherein the acrylate polymer comprises an alkyl(meth)acrylate polymer having an alkyl chain length from 2 to 4 carbons.

4. The article of claim 3 wherein the acrylate polymer comprises a butyl(meth)acrylate polymer.

5. The article of claim 1 wherein the first reactive group is an amine-reactive group.

6. The article of claim 5 wherein the first reactive group is selected from N-oxysuccinimide, isothiocyanate, bromoacetyl, epoxide, and trimethylsilane.

7. The article of claim 6 wherein the acrylate polymer is formed by copolymerizing an acrylate monomer with a monomer comprising (a) a group selected from N-oxysuccinimide, isothiocyanate, bromoacetyl, epoxide, and trimethylsilane; and (b) a vinyl group.

8. The article of claim 1 wherein 20% or less of the monomers of the acrylate polymer include first reactive groups.

9. The article of claim 1 wherein 5% or greater of the monomers of the acrylate polymer include first reactive groups.

10. The article of claim 1 wherein 5% to 20% of the monomers of the acrylate polymer include first reactive groups.

11. The article of claim 1 wherein the biomolecule comprises a biocompatible agent.

12. The article of claim 1 wherein the biomolecule comprises a polypeptide.

13. The article of claim 1 wherein the biomolecule comprises a polysaccharide.

14. The article of claim 13 wherein the biomolecule is heparin.

15. The article of claim 14 having a surface heparin activity of 5 mU/cm² or greater.

16. The article of claim 1 selected from implantable medical devices.

17. The article of claim 1 wherein the first coated layer further comprises a bioactive agent that is not coupled to the polymeric material.

18. The medical article of claim 17 wherein the bioactive agent is selected from the group consisting of anti-proliferative agents, an anti-mitotics, and antibiotics.

19. A method for providing a coating to a medical device comprising the steps of:
(a) obtaining a medical device having a coated layer comprising an acrylate polymer comprising a pendent first reactive group;
(b) disposing a coating composition on the coated layer that comprises the acrylate polymer, wherein the coating composition comprises a biomolecule, and wherein the biomolecule is reacted with the first reactive group to coupled to the biomolecule to the acrylate polymer.

20. An implantable medical device having a coating with heparin activity of 5 mU/cm² or greater, the coating comprising:
(a) a coated layer comprising an acrylate polymer comprising a pendent first reactive group; and
(b) heparin coupled to the acrylate polymer via the first reactive group.
